(19) United States
(12) Patent Application Publication
(10) Pub. No.: US 2025/0367227 A1
(43) Pub. Date: Dec. 4, 2025

(54) FOLIC ACID-MODIFIED LIPOSOME-ENCAPSULATED TILIANIN NANOCRYSTAL (FA-Lipo@Til NC), PREPARATION METHOD AND USE THEREOF

(71) Applicant: THE 1ST AFFILIATED HOSPITAL OF SHIHEZI UNIVERSITY, Shihezi City (CN)

(72) Inventors: Xinchun Wang, Shihezi City (CN); Xiangrong Song, Shihezi City (CN); Mengran Guo, Shihezi City (CN); Yongjun Gu, Shihezi City (CN); Min Sun, Shihezi City (CN); Chuansheng Huang, Shihezi City (CN)

(21) Appl. No.: 19/029,955

(22) Filed: Jan. 17, 2025

(30) Foreign Application Priority Data
May 29, 2024 (CN) 202410685366.0

(51) Int. Cl. A61K 31/7048; A61K 9/127; A61K 31/519; A61K 47/10; A61K 47/24; A61K 47/28; A61P 37/06

(52) U.S. Cl. CPC A61K 31/7048; A61K 9/127; A61K 31/519; A61K 47/10; A61K 47/24; A61K 47/28; A61P 37/06

(57) ABSTRACT

A folic acid-modified liposome-encapsulated tilianin nanocrystal (FA-Lipo@Til NC), and a preparation method and use thereof are provided. The FA-Lipo@Til NC includes a tilianin nanocrystal (Til NC) composition and a folic acid-modified phospholipid (FA-Lipo) bilayer encapsulated on a surface of the Til NC; where raw materials of the FA-Lipo bilayer include phospholipid, cholesterol (Chol), a methoxy poly(ethylene glycol)-cholesterol conjugate (mPEG-Chol), and a folic acid (FA) compound. The FA-Lipo@Til NC has a high drug loading capacity and a desirable stability. The inhibition of a Til dissolution behavior improves a penetration efficiency of the Til NC composition in intestinal mucus and an affinity with intestinal epithelial cells, thus effectively improving bioavailability of the Til.

### CROSS-REFERENCE TO RELATED APPLICATION

This patent application claims the benefit and priority of Chinese Patent Application No. 2024106853660 filed with the China National Intellectual Property Administration on May 29, 2024, the disclosure of which is incorporated by reference herein in its entirety as part of the present application.

### TECHNICAL FIELD

The present disclosure relates to the technical field of medicine, and in particular to a folic acid-modified liposome-encapsulated tilianin nanocrystal (FA-Lipo@Til NC), and a preparation method and use thereof.

### BACKGROUND

Cardiovascular diseases (CVDs), mainly including ischemic heart disease, cerebrovascular disease, and peripheral arterial disease, have an increasing prevalence worldwide. The development of effective treatments and preventive measures against infectious agents (such as antibiotics, vaccines, and modern hygiene concepts) for CVDs has replaced infectious diseases, making the CVDs a leading cause of death worldwide. Atherosclerosis is the most common form of CVDs.

The development and widespread use of effective measures to prevent or treat CVDs (including cholesterol-lowering and antihypertensive drugs, bypass surgery, and percutaneous vascular interventions) has led to a significant reduction in mortality from CVDs in industrialized countries. Despite this, CVDs remain the leading cause of death worldwide. The persistently high CVD mortality may be due to the fact that 30% to 50% of CVD patients are not exposed to traditional risk factors. Clinically, classical therapies for the atherosclerosis mainly include lipid-lowering drugs, aspirin, nitroglycerin, and fibrates, as well as recent molecular and genetic interventions exemplified by PCSK9 monoclonal antibodies and PCSK9 siRNA. The most famous lipid-lowering drugs are statins, typically including atorvastatin, simvastatin, rosuvastatin, and pravastatin. Such drugs can effectively reduce the risk of atherosclerotic CVDs and inhibit the progression of atherosclerosis by lowering the levels of plasma low-density lipoprotein cholesterol and triglycerides and increasing the levels of plasma high-density lipoprotein cholesterol. However, the incidence of major adverse cardiovascular events remains high, reaching 20% in the first three years after an acute coronary syndrome. There is also growing evidence that statin therapy demonstrates many safety issues, such as non-allergic rhinitis, rhabdomyolysis, and hepatotoxicity. An increased risk of diabetes with statin application has also been reported in some clinical trials. In addition to statins, aspirin medications are known for their potent antithrombotic and anti-inflammatory properties. Aspirin can inhibit atherosclerotic cardiovascular events by inhibiting the expression of cyclooxygenase-1 and proinflammatory cytokines. However, aspirin application is associated with some side effects, including gastrointestinal damages and allergic reactions. In summary, the above phenomena indicate that currently available treatment options are insufficiently protective and lack specificity, and novel approaches are required to overcome the shortcomings and side effects of conventional drugs.

Macrophages are involved in all stages of the occurrence and development of atherosclerosis, from plaque initiation to transition toward vulnerable plaque, and are considered important therapeutic targets. Macrophages in atherosclerotic lesions are mainly composed of short-lived monocyte-derived macrophages and vascular-resident macrophages. Circulating monocytes are recruited by a series of cytokines in the local microenvironment and migrate into surrounding tissues to further differentiate into the macrophages. Notably, the contribution of vascular-resident macrophages to atherosclerosis may decrease with age due to their diminished self-renewal capacity and then be replaced by monocyte-derived macrophages. Macrophages are key defenders against immune danger signals in the early stages of atherosclerosis. The activated immune system promotes the repair of damaged tissues, inhibits the development of atherosclerosis, and guides macrophages to conduct endocytosis by inducing the secretion of anti-inflammatory factors. In advanced and advanced atherosclerotic lesions, the macrophages are important executors of deteriorating plaque stability and further accelerate plaque rupture. The endocytic capacity of macrophages is severely impaired when they are in a high-risk internal environment for a long time, further increasing the burden of plaques. Accumulating evidence indicates that macrophage polarization largely influences plaque formation. The strong phagocytic effect of pro-inflammatory macrophages (M1 macrophages) on atherogenic factors greatly weakens the stability of plaques, ultimately leading to the formation of vulnerable plaques. In contrast, anti-inflammatory macrophage subsets (M2 macrophages) can improve plaque stability and delay the progression of vulnerable plaques. In addition, macrophage polarization is closely related to the clearance of apoptotic cells (efferocytosis). The phagocytic clearance of apoptotic cells by M1 macrophages is much weaker than that by M2 macrophages, which increases the risk of apoptotic cells evolving into necrotic cells and ultimately leads to the expansion of necrotic core area. Folic acid (FA) receptors are highly expressed on the surface of macrophages. Studies have shown that drug carriers containing FA can bind to FA receptors on the cell surface and allow the drugs carried in the carrier to enter the macrophages to take effect, thereby achieving treatment. Therefore, macrophages can be considered as a target for cellular therapy of atherosclerosis, and modulation of function on the macrophages can lead to attenuated inflammation and the development of atheroprotective conditions.

Tilianin (Til) is the main active substance in Xinjiang ethnic medicine Dracocephalum moldavica, has a chemical structure shown in Formula 1, and belongs to a flavonoid monomer compound. Til has a wide range of biological activities, including anti-diabetic, anti-inflammatory, antioxidant, anti-depressant, cardioprotective, and neuroprotective effects. The basic mechanisms of Til in protecting against atherosclerosis include improving inflammatory response, lowering cholesterol levels, regulating lipid metabolism, and reducing oxidative stress. Til shows a positive effect on macrophages and arterial endothelial cells, preventing excessive proliferation of vascular smooth muscle cells (VSMCs).

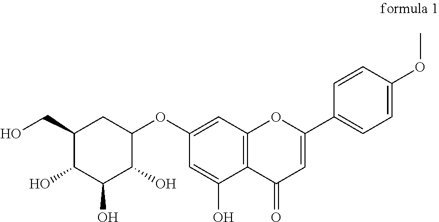 [SMILES: COc1ccc(-c2cc(=O)c3c(O)cc(OC4C[C@H](CO)[C@@H](O)[C@H](O)[C@H]4O)cc3o2)cc1]

However, despite its significant pharmacological importance as a cardioprotectant, Til is classified as a BCS class IV drug due to poor solubility in aqueous media (only 0.00157 g/L at 37° C.) and limited permeability through the gastrointestinal epithelium. The Til can generate Til monoglucuronides and acacetin monoglucuronides under the action of uridine diphosphate (UDP)-glycosyltransferase (UGT). In addition, P-glycoprotein (P-gp) and Na⁺-dependent glucose transporter 1 (SGLT1) are involved in the efflux of Til metabolites, resulting in an extremely low absolute oral bioavailability of Til (1.350±0.710) %, which seriously affects an efficacy of the drug. In order to overcome the poor water solubility and low bioavailability, some formulation strategies have been reported. These formulations have improved the solubility of Til to a certain extent, but show poor drug loading capacity and require a large amount of carrier auxiliary materials, which greatly increase toxic side effects caused by the carrier auxiliary materials. For example, CN107115321A has disclosed a Til solid lipid nanoparticle, with a drug loading capacity of about 4.4% to 6.5%. CN109875962A has disclosed an oxidation-responsive nanomicelle of Til, with a drug loading capacity of about 2.3% to 4.8%. CN102860982A has disclosed a microemulsion of Til, with a drug loading capacity of about 0.1% to 1.8%, and the microemulsion is easily affected by pH and enzymes in the gastrointestinal tract, resulting in rupture of the microemulsion, such that the preparation is unstable in the gastrointestinal tract. CHEN Xiaomin et al. and YU Ning et al. each have reported a Til solid dispersion (Preparation of Til solid dispersion and its in vivo pharmacokinetics study [J]. Chinese Traditional Patent Medicine, 2021, 43(12): 3265-3269.; Preparation of Til solid dispersion and its in vivo pharmacokinetics study [J]. Chinese Journal of Ethnomedicine and Ethnopharmacy, 2021, 30(04): 19-24+48.). Although the above two Til solid dispersions improve the water solubility of Til, the drug loading capacity of the Til solid dispersion is about 12.5% to 16.7%. In addition, the Til solid dispersion is prone to aging, which can easily lead to drug absorption during storage and in the gastrointestinal tract, resulting in instability. JUE Lili et al. have prepared a Til-PLGA block copolymer nanoparticle (Preparation of Til-PLGA block copolymer nanoparticles, in vivo intestinal perfusion in rats and in vivo pharmacokinetic study [J]. Journal of Chinese Medicinal Materials, 2020, 43(07): 1687-1691.), in which the nanoparticles have a solubility of about 20% within 3 h and a drug loading capacity of (4.79±0.12) %, indicating a poor drug loading capacity. ZENG Cheng et al. have reported a TAT-PEG-modified Til composite phospholipid liposome (Process optimization and in vitro evaluation of TAT-PEG-modified Til composite phospholipid liposome [J]. Chinese Traditional and Herbal Drugs, 2018, 49(21): 5061-5069.), where the composite phospholipid liposome has a solubility of about 40% within 3 h and a drug loading capacity of (4.93±0.28) %, indicating a poor drug loading capacity. Therefore, this liposome requires a large amount of carrier material, cannot be easily metabolized in vivo for long-term use, and is prone to cause adverse reactions. JIA Qiqi et al. have reported a Til microsphere (Preparation of Til microspheres by SPG membrane emulsification method [J]. Journal of Food Safety and Quality, 2020, 11(05): 1554-1560.), in which the microspheres have a drug loading capacity of about 1.5%, require a large amount of carrier material, cannot be easily metabolized in vivo after long-term use, and are prone to cause adverse reactions. Although these formulations can increase drug solubility to a certain extent, they generally have the problem of low drug loading capacity, which limits the application of Til and is not conducive to industrialization.

### SUMMARY

In view of this, an objective of the present disclosure is to provide a folic acid-modified liposome-encapsulated tilianin nanocrystal (FA-Lipo@Til NC), and a preparation method and use thereof. In the present disclosure, the FA-Lipo@Til NC has a high drug loading capacity and shows desirable stability, and can effectively improve the bioavailability of Til, thereby significantly improving a therapeutic effect of the Til in anti-atherosclerosis.

To achieve the above objective, the present disclosure provides the following technical solutions:

The present disclosure provides a FA-Lipo@Til NC, including a tilianin nanocrystal (Til NC) composition and a folic acid-modified phospholipid (FA-Lipo) bilayer encapsulated on a surface of the Til NC; where the Til NC composition includes Til and a stabilizer; and raw materials of the FA-Lipo bilayer include phospholipid, cholesterol (Chol), a methoxy poly(ethylene glycol)-cholesterol conjugate (mPEG-Chol), and a folic acid (FA) compound, and the FA compound includes FA and/or a FA derivative.

Preferably, the FA derivative is one or more selected from the group consisting of FA-polyethylene glycol (PEG)-Chol, 1,2-distearoyl-sn-glycero-3-phosphoethanolamine (DSPE)-PEG-FA, FA-PEG, FA-PEG-amine (NH2), FA-PEG-carboxylic acid (COOH), and FA-PEG-thiol (SH).

Preferably, the stabilizer includes a suspending agent and/or a surfactant; the suspending agent is one or more selected from the group consisting of methylcellulose (MC), hydroxypropylcellulose (HPC), hydroxypropyl methylcellulose (HPMC), polyvinyl pyrrolidone (PVP), polyvinyl alcohol (PVA), PEG, sodium carboxymethylcellulose (CMC-Na), carbomer, dextran, and sodium alginate; and the surfactant is one or more selected from the group consisting of polysorbate 20, polysorbate 80, oleic acid, lauric acid, sodium deoxycholate, sodium taurocholate, sodium glycocholate, D-α-tocopherol polyethylene glycol succinate (TPGS), sodium lauryl sulfate (SLS), sodium hexadecyl sulfate (SHS), sodium octadecyl sulfate (SOS), sodium dodecyl sulfonate (SDS), polyoxyethylene castor oil, polyoxyethylene 40 hydrogenated castor oil, poloxamer 188, poloxamer 407, cetyl trimethylammonium bromide (CTAB), PEG (15)-hydroxystearate, and glyceryl monocaprylocaprate type I.

Preferably, the FA-Lipo@Til NC has a needle shape; and the FA-Lipo@Til NC has a particle size of 10 nm to 10 μm and a drug loading capacity of 5% to 99.9%.

The present disclosure further provides a method for preparing the FA-Lipo@Til NC, including the following steps:
mixing the Til with a first organic solvent to obtain an organic phase;
mixing a stabilizer with water to obtain an aqueous phase;
mixing the organic phase with the aqueous phase, and then subjecting an obtained mixed solution to crushing and removal of the first organic solvent in sequence to obtain a suspension of the Til NC composition;
mixing the phospholipid, the Chol, the mPEG-Chol, and the FA compound with a second organic solvent, and then subjecting an obtained encapsulation layer solution to removal of the second organic solvent to obtain a milky white film; and
adding the suspension of the Til NC composition into the milky white film, and then subjecting an obtained mixture to hydration and an ultrasonic treatment in sequence to obtain the FA-Lipo@Til NC.

Preferably, the organic phase and the aqueous phase are at a volume ratio of 1:5 to 1:50; the Til in the suspension of the Til NC composition has a mass concentration of 0.01weight/volume percent (w/v %) to 80 w/v %; when the stabilizer is a suspending agent, the suspending agent in the suspension of the Til NC composition has a mass concentration of 0.01 w/v % to 20 w/v %; when the stabilizer is a surfactant, the surfactant in the suspension of the Til NC composition has a mass concentration of 0.01 w/v % to 10 w/v %; and when the stabilizer includes a suspending agent and a surfactant, the suspending agent has a mass concentration of 0.01 w/v % to 20 w/v %, and the surfactant has a mass concentration of 0.01 w/v % to 10 w/v % in the suspension of the Til NC composition.

Preferably, the phospholipid has a mass concentration of 0.05 w/v % to 20 w/v %, the Chol has a mass concentration of 0.01 w/v % to 5 w/v %, the mPEG-Chol has a mass concentration of 0.002 w/v % to 1 w/v %, and the FA compound has a mass concentration of 0.0001 w/v % to 1 w/v % in the encapsulation layer solution.

Preferably, the Til in the suspension of the Til NC composition and the phospholipid in the milky white film are at a mass ratio of 1:1 to 5:1; and the hydration is conducted for 10 min to 120 min, and the ultrasonic treatment is conducted at an ultrasonic power of 50 W to 900 W for 3 min to 120 min.

The present disclosure further provides use of the FA-Lipo@Til NC or a FA-Lipo@Til NC prepared by the preparation method in preparation of a drug for promoting generation of an anti-inflammatory macrophage and repairing efferocytosis.

Preferably, the drug is used for treating myocardial ischemia-reperfusion injury (MIRI), acute lung injury (ALI), acute kidney injury (AKI), hypertension, myocardial infarction (MI), atherosclerosis, diabetes, cancer, allergic asthma, Parkinson's disease, a non-alcoholic liver disease, vascular dementia, or an inflammatory disease; and a dosage form of the drug is selected from the group consisting of an injection, an oral solution, an ointment for external use, a transdermal patch, a gel, a capsule, a drop pill, a drop, and a spray.

The present disclosure provides a FA-Lipo@Til NC, including a Til NC composition and a FA-Lipo bilayer encapsulated on a surface of the Til NC; where the Til NC composition includes Til and a stabilizer; and raw materials of the FA-Lipo bilayer include phospholipid, Chol, an mPEG-Chol, and a FA compound, and the FA compound includes FA and/or a FA derivative. In the present disclosure, the FA-Lipo@Til NC is a drug delivery system that combines nanocrystals (NCs) (drug NCs) and liposomes (Lipos), where the NCs significantly improve the solubility of a drug by forming the drug into nanoscale crystals, and the NCs with a high drug loading capacity are encapsulated in the Lipos formed by the FA-Lipo bilayer. The drug NCs and Lipos can interact with each other through electrostatic adsorption, thereby helping to stabilize dispersion and encapsulation of the drug in the Lipos, and then further improving the drug loading capacity. Moreover, the Til NC composition is encapsulated with the FA-Lipo bilayer. On one hand, this process can inhibit the dissolution behavior of Til, improve the penetration efficiency of the Til NC composition in intestinal mucus and the affinity with the small intestinal epithelial cells, thereby increasing the possibility of the small intestinal epithelial cells to absorb NCs as a whole, and effectively improve the oral bioavailability of Til. On the other hand, the FA can specifically and efficiently target macrophages in atherosclerotic plaques, such that the Til is maintained in the atherosclerotic plaques and then endocytosed by the macrophages or distributed throughout the plaques, so as to effectively exert the anti-inflammatory, lipid-lowering, and plaque-reducing effects of Til, and significantly improve an efficacy of the Til in anti-atherosclerosis. In addition, modification to the Lipo surface (such as PEGylation) can also reduce the recognition and clearance of Lipos by the reticuloendothelial system, thus prolong circulation time of the Lipos in vivo to further improve the bioavailability of drug. The FA-Lipo@Til NC combines the advantages of NC and Lipo, has a high drug loading capacity, and can improve the solubility and stability of the drug, reduce drug degradation and leakage, and achieve sustained release and targeted delivery, thereby providing a safer and more effective research program and strategy for the clinical application of Til drugs.

The present disclosure further provides a method for preparing the FA-Lipo@Til NC. In the present disclosure, a Til NC composition is prepared using a stabilizer through a combined method of anti-solvent precipitation-crushing; and a FA-Lipo bilayer is encapsulated on a surface of the Til NC composition through a combined method of thin film hydration-ultrasonic treatment, thereby constructing a targeted drug delivery system. The preparation method has simple steps, desirable reproducibility, significantly shortened production cycle, high yield, environmental friendliness, and significant cost-effectiveness, and is suitable for large-scale industrial production. Moreover, the obtained FA-Lipo@Til NC has uniform particle size, desirable encapsulation efficiency, high drug loading capacity, and excellent stability.

The present disclosure further provides use of the FA-Lipo@Til NC or a FA-Lipo@Til NC prepared by the preparation method in preparation of a drug for promoting generation of an anti-inflammatory macrophage and repairing efferocytosis. The FA-Lipo@Til NC is used to prepare a drug for promoting the generation of anti-inflammatory macrophages and repairing efferocytosis, and has obvious therapeutic effects in the treatment of inflammatory reactions, especially in the treatment of MIRI, atherosclerosis, ALI, MI, diabetes, and hyperlipidemia.

The results of examples show that the FA-Lipo@Til NC provided by the present disclosure has the advantages of small particle size (100 nm to 300 nm), high encapsulation efficiency, and excellent drug loading capacity. Compared with the currently reported Lipo nanoformulations, the FA-Lipo@Til NC prepared according to the preferred prescription of the present disclosure (referring to Example 3 for details) has encapsulation efficiency and drug loading capacity of (91.82±0.65) % and (48.61±0.65) %, respectively, which are much higher than those of the reported Lipo nanoformulations. The release of Til in the FA-Lipo@Til NC has a sustained release effect; and in the dissolution behavior test (FIG. 9A-FIG. 9C), the release of Til in the FA-Lipo@Til NC is reduced to about 1.5% of that of Til NC. The FA-Lipo@Til NC improves the targeted accumulation in the atherosclerotic plaque site through FA targeting (referring to FIG. 17, FIG. 18A-FIG. 18C, and FIG. 19) to exert a highly effective therapeutic effect on atherosclerosis. The FA-Lipo@Til NC effectively improves the oral bioavailability of Til (FIG. 20). The FA-Lipo@Til NC can significantly increase the AUC_{0-24 h} of Til, which was 9.43, 3.63, and 1.29 times of the Crude Til, Til NCs, and Lipo@Til NCs, respectively, and the Tₘₐₓ is prolonged. Meanwhile, the FA-Lipo@Til NC can effectively inhibit the area of aortic plaques (with an inhibition rate of 25.46%), stabilize plaques, promote the transformation of macrophages into M2 type and repair efferocytosis (referring to FIG. 21A-FIG. 21C and FIG. 22), reduce inflammation and dyslipidemia (referring to FIG. 27A-FIG. 27D and FIG. 28A-FIG. 28D), reduce the area of plaques (referring to FIG. 23 and FIG. 24), and increase the stability of plaques (referring to FIG. 25 and FIG. 26), thereby achieving effective treatment of atherosclerosis.

### BRIEF DESCRIPTION OF THE DRAWINGS

FIG. 1 shows an influence of different drug-lipid ratios on particle size and encapsulation efficiency of the FA-Lipo@Til NC;

FIG. 2 shows an influence of different FA percentage contents on particle size and encapsulation efficiency of the FA-Lipo@Til NC;

FIG. 3 shows the investigation of a fluorescence intensity on the uptake of preparations with different FA percentages by RAW264.7 cells using confocal microscopy;

FIG. 4 shows an appearance of the FA-Lipo@Til NC;

FIG. 5 shows a Tyndall effect of the FA-Lipo@Til NC;

FIG. 6 shows a transmission electron microscopy (TEM) image of the FA-Lipo@Til NC;

FIG. 7A-FIG. 7B show the stability of the FA-Lipo@Til NC at 4° C. and 25° C.;

FIG. 8A-FIG. 8B show the stability of the FA-Lipo@Til NC in SGF and SIF;

FIG. 9A-FIG. 9C show a dissolution behavior of the FA-Lipo@Til NC in different media;

FIG. 10 shows the investigation of a penetration percentage of the FA-Lipo@Til NC in an agarose gel layer after 6 h;

FIG. 11A-FIG. 11B show a cumulative release amount of the FA-Lipo@Til NC across mucus in vitro and the investigation of Papp;

FIG. 12 shows a fluorescence image of the FA-Lipo@Til NC in Caco-2 cells observed by confocal laser scanning microscopy (CLSM);

FIG. 13A-FIG. 13B show a mean fluorescence intensity of the FA-Lipo@Til NC in Caco-2 cells measured by flow cytometry (FCM);

FIG. 14A-FIG. 14C show a relative uptake of the FA-Lipo@Til NC by Caco-2 cells after adding an uptake inhibitor;

FIG. 15A-FIG. 15C show a relative uptake of the FA-Lipo@Til NC by Caco-2 cells after adding an exocytosis inhibitor;

FIG. 16A-FIG. 16F show the cumulative release amount and Papp value of the FA-Lipo@Til NC containing different Til concentrations across a monolayer cell membrane over time on the Caco-2 monolayer;

FIG. 17 shows a fluorescence image of the uptake for the FA-Lipo@Til NC across the monolayer cell membrane targeting RAW264.7 detected by CLSM qualitatively;

FIG. 18A-FIG. 18C shows the statistics of a Til content taken up by RAW264.7 after the FA-Lipo@Til NC crossing the monolayer cell membrane within 4 h;

FIG. 19 shows a fluorescence image of the aorta acquired by an interactive video information system (IVIS) and a quantitative graph of fluorescence intensity, where a left side is the fluorescence image of the aorta acquired by the IVIS system and a right side is the quantitative graph of fluorescence intensity in FIG. 19;

FIG. 20 shows a plasma concentration-time curve of the FA-Lipo@Til NC after intragastric administration;

FIG. 21A-FIG. 21C show the statistics on polarization of RA W264.7 macrophages regulated by the FA-Lipo@Til NC detected by flow cytometery;

FIG. 22 shows the statistics on efferocytosis of RA W264.7 macrophages regulated by the FA-Lipo@Til NC detected by flow cytometery;

FIG. 23 shows the quantitative statistics of Oil Red O (ORO) staining area relative to plaques throughout the aorta;

FIG. 24 shows statistics for quantitative analysis of relative ORO staining areas in aortic root sections;

FIG. 25 shows statistics for quantitative analysis of H&E-stained necrotic cores in aortic root sections;

FIG. 26 shows statistics for quantitative analysis of Masson-stained collagen content in aortic root sections;

FIG. 27A-FIG. 27D show statistics of TNF-α, IL-1β, IL-6, and IL-10 expression in serum detected by ELISA kit;

FIG. 28A-FIG. 28D show statistics of TG, TC, LDL-C, and HDL-C levels in serum of atherosclerosis mice after different treatments; and

FIG. 29 shows a schematic flow chart for the preparation method of a FA-Lipo@Til NC in an example of the present disclosure.

### DETAILED DESCRIPTION OF THE EMBODIMENTS

The present disclosure provides a FA-Lipo@Til NC, including a Til NC composition and a FA-Lipo bilayer encapsulated on a surface of the Til NC; where the Til NC composition includes Til and a stabilizer; and raw materials of the FA-Lipo bilayer include phospholipid, Chol, mPEG-Chol, and a FA compound, and the FA compound includes FA and/or a FA derivative.

In the present disclosure, the FA-Lipo@Til NC has a shell-core structure.

In the present disclosure, the FA-Lipo@Til NC includes a Til NC composition, and components of the Til NC composition include Til and a stabilizer. The Til, also known as acacetin-7-glucoside, is an active flavonoid glycoside that can be extracted from a variety of medicinal plants, especially an active flavonoid glycoside obtained from Dracocephalum moldavica. Til is also found in various common medicinal plants, including Agastache rugosa, Dracocephalum tanguticum, other Dracocephalum plants, Lygodium japonicum, and red calyx Lygodium plants. These plants are found in East Asia including China, Japan, and Korea, and also found in Mexico. Til has a wide range of biological activities, including anti-diabetic, anti-inflammatory, antioxidant, anti-depressant, cardioprotective, and neuroprotective effects as report goes. In particular, Til has a significant anti-inflammatory effect, and can inhibit the expression of endothelial nitric oxide synthase (eNOs), proinflammatory cytokines dependent on nuclear factor-κB (NF-κB), tumor necrosis factor-α (TNF-α), interleukin (IL)-1β, class A scavenger receptors, intracellular & intercellular adhesion molecules, vascular cell adhesion molecules, matrix metalloproteinase (MMP)-2, MMP-9, monocyte chemoattractant protein (MCP) 1, and proinflammatory factors, thereby exerting an anti-inflammatory effect.

In the present disclosure, the stabilizer preferably includes a suspending agent and/or a surfactant, preferably includes the suspending agent and the surfactant. The suspending agent is preferably one or more selected from the group consisting of MC, HPC, HPMC, PVP, PVA, PEG, CMC-Na, carbomer, dextran, and sodium alginate, more preferably the PVA; and the surfactant is one or more selected from the group consisting of polysorbate 20 (Tween 20), polysorbate 80 (Tween 80), oleic acid, lauric acid, sodium deoxycholate, sodium taurocholate, sodium glycocholate, TPGS, SLS (also known as SDS), SHS, SOS, SDS, polyoxyethylene castor oil (Cremophor EL), polyoxyethylene 40 hydrogenated castor oil (Cremophor RH40), poloxamer 188 (F68), poloxamer 407 (F127), CTAB, PEG (15)-hydroxystearate, and glyceryl monocaprylocaprate type I, more preferably the TPGS.

In the present disclosure, the FA-Lipo@Til NC includes a FA-Lipo bilayer encapsulated on a surface of the Til NC composition. Raw materials of the FA-Lipo bilayer include phospholipid, Chol, mPEG-Chol, and a FA compound. The phospholipid preferably includes one or more of a neutral phospholipid, a negatively charged phospholipid, and a positively charged phospholipid. The neutral phospholipid is preferably one or more of soybean lecithin, dipalmitoyl choline, distearoyl choline, and dimyristoyl phosphatidyl choline. The negatively charged phospholipid is preferably one or more of phosphatidic acid, phosphatidylglycerol, and phosphatidylinositol. The positively charged phospholipid is preferably a stearylamide Chol derivative. In an example, the phospholipid is preferably the soybean lecithin. The mPEG-Chol is preferably one or more of mPEG100-Chol, mPEG500-Chol, mPEG1000-Chol, mPEG2000-Chol, mPEG5000-Chol, and mPEG7000-Chol, more preferably the mPEG2000-Chol. The FA compound includes FA and/or a FA derivative, and the FA derivative preferably includes one or more of FA-PEG-Chol, DSPE-PEG-FA, FA-PEG, FA-PEG-NH2, FA-PEG-COOH, and FA-PEG-SH. The FA-PEG-Chol is preferably FA-PEG2000-Chol, the DSPE-PEG-FA is preferably DSPE-PEG2000-FA, the FA-PEG is preferably PEG2000-FA, the FA-PEG-NH2 is preferably FA-PEG2000-NH2, the FA-PEG-COOH is preferably FA-PEG2000-COOH, and the FA-PEG-SH is preferably FA-PEG2000-SH. In an example, the FA derivative is preferably the FA-PEG2000-Chol.

In the present disclosure, the phospholipid as a main component of Lipo has an amphiphilic structure, with one end being hydrophilic and the other end being hydrophobic. This property enables the phospholipid to form a bilayer membrane as a skeleton of Lipo; the phospholipid can not only maintain the structural stability of Lipo, but also contain water-soluble substances; in addition, the phospholipid can also regulate fat metabolism, maintain the normal structure of cell membranes, and prevent atherosclerosis. The main function of Chol is to regulate the fluidity of the phospholipid bilayer membrane, reduce membrane permeability, and reduce drug leakage; Chol can also maintain a certain flexibility of the lipid membrane and enhance the ability of Lipo vesicles to resist changes in external conditions; in addition, Chol also affects the particle size, oxidative stability, and physical stability of Lipo. The mPEG-Chol is a hydrophilic lipid material that can enhance the stability of Lipo, prolong its circulation time in vivo, and reduce the absorption and clearance of Lipo; this effect is achieved through the introduction of mPEG chains, which can reduce the interaction between Lipo and serum proteins, reduce immunogenicity, and thus prolong the circulation time of the drug in vivo; in addition, mPEG-Chol can also improve the anti-serum protein adsorption capacity and biocompatibility of Lipo, giving a wider application prospect in drug delivery. The FA compound is targeted molecules that can specifically bind to FA receptors on the surface of macrophages in atherosclerotic plaques; the FA compound is used as a targeting ligand and then combined with Lipo through covalent or non-covalent bonds to form a FA-Lipo bilayer to achieve targeted drug delivery; this targeted drug delivery can increase the concentration of drugs in atherosclerotic plaques, thereby improving the efficacy of drugs. At the same time, after modification with PEG and FA, Lipo can better encapsulate and protect drugs, reduce drug degradation and leakage, and thus improve the bioavailability and efficacy of drugs. In addition, these modifications can also change the surface properties of Lipo, making it easier to penetrate the cell membrane and enter the cell, thus further improving the drug delivery effect.

In the present disclosure, the FA-Lipo@Til NC has a needle shape and shiny; compared with Til drugs that exist in round or disc shapes, needle-shaped Lipo NC is easier to stay in atherosclerotic plaques under the action of overcoming blood flow shear force. Moreover, FA functional modification can enable Til to target macrophages in atherosclerotic plaques; under a dual action, it is easier for the drug to exert a better therapeutic effect in atherosclerotic plaques. The FA-Lipo@Til NC has a particle size of preferably 10 nm to 10 μm, more preferably 10 nm to 500 nm, and even more preferably 10 nm to 200 nm; the FA-Lipo@Til NC has a drug loading capacity of preferably 5% to 99.9%, more preferably 10% to 70%, and further optimized to 30% to 60%.

In the present disclosure, the FA-Lipo@Til NC has a high drug loading capacity and desirable stability. By inhibiting the dissolution behavior of Til in the Til NC composition, the penetration efficiency of the Til NC composition in intestinal mucus and the affinity with the small intestinal epithelial cells are both improved, thereby increasing the possibility of the small intestinal epithelial cells to take up NCs as a whole, and then effectively improving the oral bioavailability of Til. Moreover, the FA can specifically and efficiently target macrophages in atherosclerotic plaques, such that the Til is maintained in the atherosclerotic plaques and then endocytosed by the macrophages or distributed throughout the plaques, thereby effectively exerting the anti-inflammatory, lipid-lowering, and plaque-reducing effects of Til, and then significantly improving the efficacy of Til in anti-atherosclerosis.

The present disclosure further provides a method for preparing the FA-Lipo@Til NC, including the following steps:
mixing the Til with a first organic solvent to obtain an organic phase;
mixing a stabilizer with water to obtain an aqueous phase;
mixing the organic phase with the aqueous phase, and then subjecting an obtained mixed solution to crushing and removal of the first organic solvent in sequence to obtain a suspension of the Til NC composition;
mixing the phospholipid, the Chol, the mPEG-Chol, and the FA compound with a second organic solvent, and then subjecting an obtained encapsulation layer solution to removal of the second organic solvent to obtain a milky white film; and
adding the suspension of the Til NC composition into the milky white film, and then subjecting an obtained mixture to hydration and an ultrasonic treatment in sequence to obtain the FA-Lipo@Til NC.

In the present disclosure, unless otherwise specified, all raw materials are commercially available products well known to persons skilled in the art.

In the present disclosure, the Til is mixed with a first organic solvent to obtain an organic phase. The first organic solvent preferably includes one or more of methanol (MeOH), ethanol (EtOH), acetonitrile (AC), dimethyl sulfoxide (DMSO), N,N-dimethylformamide (DMF), dimethylacetamide (DMA), N-methylpyrrolidone (NMP), tetrahydrofuran (THF), acetone, propylene glycol, and isopropanol, more preferably one or two thereof; when the first organic solvent preferably includes two of the above solvents, the two solvents are at a volume ratio of preferably 1:1. Since different organic solvents have different diffusion coefficients in water, which may lead to different precipitation rates of Til NC in the aqueous phase, and in turn affects the particle size of Til NC. The types of organic solvent can be screened based on the particle size and particle size distribution coefficient. In an example, the first organic solvent includes preferably DMF and anhydrous ethanol, and the DMF and the anhydrous ethanol are at a volume ratio of preferably 1:1. The mixing is based on dissolving Til in the first organic solvent.

In the present disclosure, the stabilizer is mixed with water to obtain an aqueous phase. The stabilizer is the same as the stabilizer described in the above technical solutions, and will not be described in detail here. The mixing is based on dissolving the stabilizer in water.

In the present disclosure, the organic phase is mixed with the aqueous phase, and then an obtained mixed solution is subjected to crushing and removal of the first organic solvent in sequence to obtain a suspension of the Til NC composition. The organic phase and the aqueous phase are at a volume ratio of preferably 1:5 to 1:50, more preferably 1:10 to 1:30, and most preferably 1:20. Preferably, the organic phase is mixed rapidly with the aqueous phase.

In the present disclosure, a crushing method preferably includes probe sonication, high-pressure homogenization, wet grinding, or micro jet. The probe sonication, also known as ultrasonication, refers to the use of shock waves and shear forces caused by a cavitation effect of ultrasound in liquid to control crystal formation or break up particles to obtain the NCs. The high-pressure homogenization refers to a process of passing drug crystals through ultra-fine holes at high pressure, such that they are subjected to multiple high-speed shearing and impact forces in a highly short time, thereby breaking the crystalline particles. The wet grinding refers to the generation of a shear force of an extrusion effect on the surface of crystalline particles by stirring and rolling the grinding medium in a liquid medium, thereby breaking the crystalline particles. The micro jet refers to the formation of ultrasonic flow rate when the pressure drops sharply when passing through a valve core with a tiny aperture under the action of ultra-high pressure, causing particle impact, cavitation, and shear force in the fluid, thereby breaking the crystalline particles. In an example, the crushing method is preferably the probe sonication, and the probe sonication is conducted at an ultrasound power of 50 W to 900 W, preferably 130 W to 150 W for 3 min to 120 min, preferably 20 min to 25 min preferably in an ice bath.

In the present disclosure, the Til NC composition is prepared by the anti-solvent precipitation-crushing combined method. In preparing the NCs by anti-solvent precipitation-crushing combined method, the drug and the stabilizer are present in a dissolved state in a good solvent and then precipitate to form NCs with the addition of the anti-solvent. In the crushing stage, if the probe sonication is adopted, the cavitation effect and mechanical vibration generated by the ultrasound can promote the uniform mixing and rapid precipitation of the drug molecules and the stabilizer molecules, thereby obtaining smaller and more uniform NCs. The stabilizer stabilizes the size and dispersibility of the NCs by adsorbing on the surface of the drug particles or forming a complex with the drug molecules.

In the present disclosure, a process for removal of the organic solvent is preferably dialysis; the dialysis preferably includes: placing a suspension of the composition obtained after the crushing in a dialysis bag, and then placing the dialysis bag in pure water for dialysis to remove the organic solvent to obtain a suspension of the Til NC composition; where the dialysis bag has a molecular weight of preferably 3,500 MW, and the dialysis is conducted for preferably 24 h.

In the present disclosure, the suspension of the Til NC composition includes Til, a stabilizer, and water. The Til in the suspension of the Til NC composition has a mass concentration of preferably 0.01 w/v % to 80 w/v %, more preferably 0.01 w/v % to 40 w/v %, even more preferably 0.01 w/v % to 1 w/v %, and most preferably 0.3 w/v % to 0.6 w/v %. When the stabilizer is a suspending agent, the suspending agent in the suspension of the Til NC composition has a mass concentration of preferably 0.01 w/v % to 20 w/v %. When the stabilizer is a surfactant, the surfactant in the suspension of the Til NC composition has a mass concentration of preferably 0.01 w/v % to 10 w/v %. When the stabilizer includes a suspending agent and a surfactant, in the suspension of the Til NC composition, the suspending agent has a mass concentration of preferably 0.01 w/v % to 20 w/v %, more preferably 0.1 w/v % to 2.0 w/v %, and specifically preferably 0.2 w/v %, 0.6 w/v %, 0.05 w/v %, 0.175 w/v %, or 0.3 w/v %; the surfactant has a mass concentration of preferably 0.01 w/v % to 10 w/v %, more preferably 0.01w/v % to 1.0 w/v %, and specifically preferably 0.8 w/v %, 0.1 w/v %, 0.05 w/v %, 0.12 w/v %, or 0.08 w/v %. The “w/v %” is a mass concentration unit, which indicates a mass (g) of a substance per 100 mL of a solution. The suspending agent can be adsorbed on the surface of Til NC to form a steric stabilizing effect, affecting the nucleation and crystal growth of crystals, and the type, molecular weight, and mass volume concentration of the suspending agent in the suspension can be adjusted; the type, molecular weight, and mass volume concentration of the surfactant affect the nucleation and crystal growth of crystals, and the type, molecular weight, and mass volume concentration of the surfactant in the suspension can be adjusted.

In the present disclosure, the Til NC composition in the suspension has a particle size of preferably 90 nm to 110 nm.

In the present disclosure, the phospholipid, the Chol, the mPEG-Chol, and the FA compound are mixed with a second organic solvent, and then an obtained encapsulation layer solution is subjected to removal of the second organic solvent to obtain a milky white film. The Fa compound is the same as the FA compound described in the above technical solutions, and will not be repeated here.

In the present disclosure, the second organic solvent preferably includes one or more of anhydrous ethanol, chloroform, methanol, THF, acetone, isopropanol, and carbon tetrachloride, more preferably the anhydrous ethanol.

In the encapsulation layer solution of the present disclosure, the phospholipid has a mass concentration of preferably 0.05 w/v % to 20 w/v %, more preferably 0.05 w/v % to 5 w/v %, and even more preferably 0.1 w/v %; the Chol has a mass concentration of 0.01 w/v % to 5 w/v %, more preferably 0.01 w/v % to 1.25 w/v %, and even more preferably 0.025 w/v %; the mPEG-Chol has a mass concentration of preferably 0.002 w/v % to 1 w/v %, more preferably 0.002 w/v % to 0.5 w/v %, and even more preferably 0.0041 w/v %; the FA compound has a mass concentration of preferably 0.0001 w/v % to 1 w/v %, more preferably 0.0001 w/v % to 0.5 w/v %, and even more preferably 0.00041 w/v %, 0.0008 w/v %, 0.0017 w/v %, 0.0041 w/v %, or 0.008 w/v %. A mass percentage of the FA compound is preferably 0.1% to 15%, more preferably 0.3% to 10%, specifically 0.3%, 0.6%, 1.2%, 3%, or 6% of a mass of the total lipid components (phospholipid, Chol, mPEG-Chol, and FA compound). The type, molecular weight, and proportion of the FA compound in the total lipid components all affect its uptake by macrophages.

In the present disclosure, a process for removal of the organic solvent is preferably vacuum evaporation, and the vacuum evaporation is conducted at preferably 30° C. to 70° C., more preferably 40° C. to 60° C. for preferably 10 min to 120 min, more preferably 30 min to 80 min. After the removal of organic solvent, a milky white film is obtained.

In the present disclosure, the suspension of the Til NC composition is added into the milky white film, and then an obtained mixture is subjected to hydration and an ultrasonic treatment in sequence to obtain the FA-Lipo@Til NC.

In the present disclosure, the Til NC composition is prepared using a suspending agent and/or surfactant through an anti-solvent precipitation-crushing combined method; and the FA-Lipo bilayer is encapsulated on a surface of the Til NC composition through a combined method of thin film hydration-ultrasonic treatment, thereby constructing the FA-Lipo@Til NC. The combined method of thin film hydration-ultrasonic treatment is to conduct rotary evaporation on the organic solvent under vacuum to form a thin film of lipid in a flask, and then add an aqueous phase solution to hydrate the lipid film to form a multilayer Lipo suspension; it is possible to further ultrasonically reduce the particle size to obtain a small single-chamber Lipo.

In the present disclosure, the Til in the suspension of the Til NC composition and the phospholipid in the milky white film are at a mass ratio of preferably 1:1 to 5:1, more preferably 1:1 to 3:1. The hydration is conducted for preferably 10 min to 120 min, more preferably 20 min to 60 min, and can be done at a room temperature; the ultrasonic treatment is conducted at a power of preferably 50 W to 900 W, more preferably 90 W to 120 W for preferably 3 min to 120 min, more preferably 10 min to 60 min; the ultrasonic treatment is preferably completed by probe sonication, and the probe sonication is preferably conducted in an ice bath.

In the present disclosure, the FA-Lipo@Til NC has an encapsulation efficiency of preferably 5% to 99.9%, more preferably 50% to 99.9%, even more preferably 70% to 99.9%, and most preferably 80% to 99.9%. The encapsulation efficiency is calculated according to the following Formula 1:

Encapsulation efficiency=((total amount of drug-total amount of drug in lower supernatant)/total amount of drug)×100%, Formula 1; where

In Formula 1, the total amount of drug is a total mass of Til added during preparation, and the total mass of Til is determined by high-performance liquid chromatography (HPLC); the total amount of drug in lower supernatant is a total mass of Til not encapsulated by Lipo membrane, and the total amount of drug in lower supernatant is determined by separating the Til encapsulated in FA-Lipo@Til NC via high-speed centrifugation, re-dissolving the drug in the lower supernatant with methanol, and then determining by HPLC.

In the present disclosure, the FA-Lipo@Til NC has a drug loading capacity of preferably 5% to 99.9%, more preferably 10% to 70%, further optimized to 20% to 60%, and most preferably 30% to 60%. The drug loading capacity is calculated according to the following Formula 2:

Drug loading capacity=((total amount of drug−total amount of drug in lower supernatant)/total mass of raw materials)×100%, Formula 2; where

In Formula 2, the total mass of raw materials is a total mass of Til, phospholipid, Chol, mPEG-Chol, and FA compound added during preparation; the total amount of drug is a total mass of Til added during preparation, and the total mass of Til is determined by HPLC; the total amount of drug in lower supernatant is a total mass of Til not encapsulated by Lipo membrane, and the total amount of drug in lower supernatant is determined by separating the Til encapsulated in FA-Lipo@Til NC via high-speed centrifugation, re-dissolving the drug in the lower supernatant with methanol, and then determining by HPLC.

In the present disclosure, the preparation method has the advantages of simple process, desirable reproducibility, and easy industrial transformation.

The present disclosure further provides use of the FA-Lipo@Til NC or a FA-Lipo@Til NC prepared by the preparation method in preparation of a drug for promoting generation of an anti-inflammatory macrophage and repairing efferocytosis.

In the present disclosure, the FA-Lipo@Til NC can promote the polarization of macrophages into the anti-inflammatory M2 type and repair the macrophage efferocytosis, while inhibiting the production of reactive oxygen species and pro-inflammatory factors, thereby exerting an anti-inflammatory effect.

In the present disclosure, the drug is preferably used for treating MIRI, ALI, AKI, hypertension, MI, atherosclerosis, diabetes, cancer, allergic asthma, Parkinson's disease, a non-alcoholic liver disease, vascular dementia, or an inflammatory disease; and the inflammatory disease preferably includes but is not limited to inflammatory bowel disease or arthritis. In an example, the drug is preferably used for treating atherosclerosis. A dosage form of the drug is preferably selected from the group consisting of an injection (such as intravenous injection), an oral solution, an external ointment, a transdermal patch (i.e., transdermal administration), a gel, a capsule, a drop pill, a drop, and a spray (such as a nasal spray). In an example, the drug is preferably administered orally, that is, an oral atherosclerotic plaque-targeted FA-Lipo@Til NC is constructed in an example of the present disclosure. The FA-Lipo@Til NC can improve the mucus penetration efficiency and transmembrane transport ability of Til after oral administration, and can target the macrophages in atherosclerotic plaques, such that Til is mainly concentrated in the aortic plaques and specifically targets the plaque cells, thereby reducing local or systemic toxic and side effects of the body and improving the bioavailability of Til.

To further explain the present disclosure, the FA-Lipo@Til NC, and the preparation method and the use thereof provided in the present disclosure will be described in detail below in conjunction with examples which, however, should not be interpreted as limitations to the protection scope of the present disclosure.

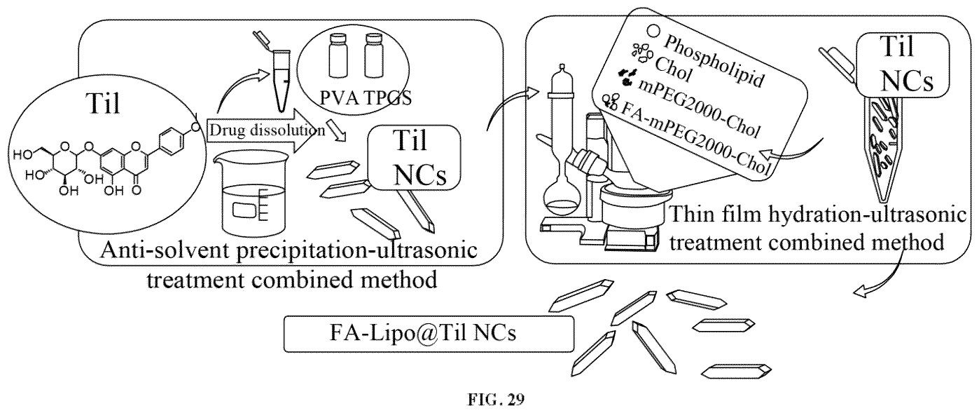

In the following examples, the preparation is conducted according to the preparation flow chart of a FA-Lipo@Til NC (also referred to as FA-modified Lipo NC containing Til) provided in FIG. 29.

### Example 1

6 mg of Til was dissolved in 100 μL of organic solvent of anhydrous ethanol: DMF (at a volume ratio of 1:1) to obtain an organic phase, 3 mg of PVA and 0.8 mg of TPGS were dissolved in 2 mL of water to obtain an aqueous phase, the organic phase and the aqueous phase were quickly mixed and subjected to probe sonication at 130 W for 25 min using an ultrasonic cell disruptor in an ice bath, and then the organic solvent was removed by dialysis to obtain a suspension of Til NCs with a particle size of 99.9 nm±1.66 nm and a PDI of 0.31±0.01. The Til NCs were used as a drug-carrying core, soybean lecithin was used as a main lipid, and Chol was used as a main component, a FA-modified phospholipid bilayer was constructed on the surface of Til NCs by a combined method of thin film hydration-ultrasonic treatment to form FA-Lipo@Til NCs with a shell-core structure: the soybean lecithin, Chol, mPEG2000-Chol, and FA-PEG2000-Chol had masses of 6 mg, 1.5 mg, 0.25 mg, and 0.25 mg, respectively, and were dissolved in 6 mL of anhydrous ethanol, the anhydrous ethanol in the round-bottom flask was removed by rotary evaporation in a 60° C. water bath, and then the auxiliary materials were evenly spread on the bottom of the flask to form a dense lipid film by rotary evaporation for 30min. 2 mL of suspension of Til NCs, containing 6 mg of drug (Til), was poured into the round-bottom flask forming the lipid film to allow hydration at room temperature for 30 min. The samples were subjected to ultrasonic treatment at 120 W for 10 min in an ice bath. Finally, FA-Lipo@Til NCs were obtained, with a particle size of 135.43 nm±0.15 nm, a PDI of 0.51±0.01, an encapsulation efficiency of (92.02+0.50) %, and a drug loading capacity of (39.44±0.50) %.

### Example 2

7.5 mg of Til was dissolved in 100 μL of an organic solvent of anhydrous ethanol: DMF (at a volume ratio of 1:1) to obtain an organic phase, 3 mg of PVA and 0.8 mg of TPGS were dissolved in 2 mL of water to obtain an aqueous phase, the organic phase and the aqueous phase were quickly mixed and subjected to probe sonication at 130 W for 25 min using an ultrasonic cell disruptor in an ice bath, and then the organic solvent was removed by dialysis to obtain a suspension of Til NCs with a particle size of 103.45 nm±0.23 nm and a PDI of 0.45+0.01. The Til NCs were used as a drug-carrying core, soybean lecithin was used as a main lipid, and Chol was used as a main component, a FA-modified phospholipid bilayer was constructed on the surface of Til NCs by a combined method of thin film hydration-ultrasonic treatment to form FA-Lipo@Til NCs with a shell-core structure: the soybean lecithin, Chol, mPEG2000-Chol, and FA-PEG2000-Chol had masses of 6 mg, 1.5 mg, 0.25 mg, and 0.25 mg, respectively, and were dissolved in 6 mL of anhydrous ethanol, the anhydrous ethanol in the round-bottom flask was removed by rotary evaporation in a 60° C. water bath, and then the auxiliary materials were evenly spread on the bottom of the flask to form a dense lipid film by rotary evaporation for 30 min. 2 mL of suspension of Til NCs, containing 7.5 mg of drug (Til), was poured into the round-bottom flask forming the lipid film to allow hydration at room temperature for 30 min. The samples were subjected to ultrasonic treatment at 120 W for 10 min in an ice bath. Finally, FA-Lipo@Til NCs were obtained, with a particle size of 146.03 nm±0.64 nm, a PDI of 0.55±0.02, an encapsulation efficiency of (91.95±0.49) %, and a drug loading capacity of (44.49±0.49) %.

### Example 3

9 mg of Til was dissolved in 100 μL of an organic solvent of anhydrous ethanol: DMF (at a volume ratio of 1:1) to obtain an organic phase, 3 mg of PVA and 0.8 mg of TPGS were dissolved in 2 mL of water to obtain an aqueous phase, the organic phase and the aqueous phase were quickly mixed and subjected to probe sonication at 130 W for 25 min using an ultrasonic cell disruptor in an ice bath, and then the organic solvent was removed by dialysis to obtain a suspension of Til NCs with a particle size of 103.56 nm±0.51 nm and a PDI of 0.55±0.02. The Til NCs were used as a drug-carrying core, soybean lecithin was used as a main lipid, and Chol was used as a main component, a FA-modified phospholipid bilayer was constructed on the surface of Til NCs by a combined method of thin film hydration-ultrasonic treatment to form FA-Lipo@Til NCs with a shell-core structure: the soybean lecithin, Chol, mPEG2000-Chol, and FA-PEG2000-Chol had masses of 6 mg, 1.5 mg, 0.25 mg, and 0.25 mg, respectively, and were dissolved in 6 mL of anhydrous ethanol, the anhydrous ethanol in the round-bottom flask was removed by rotary evaporation in a 60° C. water bath, and then the auxiliary materials were evenly spread on the bottom of the flask to form a dense lipid film by rotary evaporation for 30 min. 2 mL of suspension of Til NCs, containing 9 mg of drug (Til), was poured into the round-bottom flask forming the lipid film to allow hydration at room temperature for 30 min. The samples were subjected to ultrasonic treatment at 120 W for 10 min in an ice bath. Finally, FA-Lipo@Til NCs were obtained, with a particle size of 147.46 nm±1.55 nm, a PDI of 0.57±0.02, an encapsulation efficiency of (91.82±0.65) %, and a drug loading capacity of (48.61±0.65) %.

### Example 4

10.5 mg of Til was dissolved in 100 μL of an organic solvent of anhydrous ethanol: DMF (at a volume ratio of 1:1) to obtain an organic phase, 3 mg of PVA and 0.8 mg of TPGS were dissolved in 2 mL of water to obtain an aqueous phase, the organic phase and the aqueous phase were quickly mixed and subjected to probe sonication at 130 W for 25 min using an ultrasonic cell disruptor in an ice bath, and then the organic solvent was removed by dialysis to obtain a suspension of Til NCs with a particle size of 101.26 nm±0.18 nm and a PDI of 0.54±0.02. The Til NCs were used as a drug-carrying core, soybean lecithin was used as a main lipid, and Chol was used as a main component, a FA-modified phospholipid bilayer was constructed on the surface of Til NCs by a combined method of thin film hydration-ultrasonic treatment to form FA-Lipo@Til NCs with a shell-core structure: the soybean lecithin, Chol, mPEG2000-Chol, and FA-PEG2000-Chol had masses of 6 mg, 1.5 mg, 0.25 mg, and 0.25 mg, respectively, and were dissolved in 6 mL of anhydrous ethanol, the anhydrous ethanol in the round-bottom flask was removed by rotary evaporation in a 60° C. water bath, and then the auxiliary materials were evenly spread on the bottom of the flask to form a dense lipid film by rotary evaporation for 30 min. 2 mL of suspension of Til NCs, containing 10.5 mg of drug (Til), was poured into the round-bottom flask forming the lipid film to allow hydration at room temperature for 30 min. The samples were subjected to ultrasonic treatment at 120 W for 10 min in an ice bath. Finally, FA-Lipo@Til NCs were obtained, with a particle size of 155.23 nm±0.60 nm, a PDI of 0.52±0.01, an encapsulation efficiency of (89.94±0.79) %, and a drug loading capacity of (50.74±0.79) %.

### Example 5

12 mg of Til was dissolved in 100 μL of an organic solvent of anhydrous ethanol: DMF (at a volume ratio of 1:1) to obtain an organic phase, 3 mg of PVA and 0.8 mg of TPGS were dissolved in 2 mL of water to obtain an aqueous phase, the organic phase and the aqueous phase were quickly mixed and subjected to probe sonication at 130 W for 25 min using an ultrasonic cell disruptor in an ice bath, and then the organic solvent was removed by dialysis to obtain a suspension of Til NCs with a particle size of 105.63 nm±0.27 nm and a PDI of 0.59±0.23. The Til NCs were used as a drug-carrying core, soybean lecithin was used as a main lipid, and Chol was used as a main component, a FA-modified phospholipid bilayer was constructed on the surface of Til NCs by a combined method of thin film hydration-ultrasonic treatment to form FA-Lipo@Til NCs with a shell-core structure: the soybean lecithin, Chol, mPEG2000-Chol, and FA-PEG2000-Chol had masses of 6 mg, 1.5 mg, 0.25 mg, and 0.25 mg, respectively, and were dissolved in 6 mL of anhydrous ethanol, the anhydrous ethanol in the round-bottom flask was removed by rotary evaporation in a 60° C. water bath, and then the auxiliary materials were evenly spread on the bottom of the flask to form a dense lipid film by rotary evaporation for 30 min. 2 mL of suspension of Til NCs, containing 12 mg of drug (Til), was poured into the round-bottom flask forming the lipid film to allow hydration at room temperature for 30 min. The samples were subjected to ultrasonic treatment at 120 W for 10 min in an ice bath. Finally, FA-Lipo@Til NCs were obtained, with a particle size of 165.23 nm±0.66 nm, a PDI of 0.56±0.05, an encapsulation efficiency of (87.32±0.53) %, and a drug loading capacity of (52.20±0.53) %.

### Example 6

9 mg of Til was dissolved in 100 μL of an organic solvent of anhydrous ethanol: DMF (at a volume ratio of 1:1) to obtain an organic phase, 3 mg of PVA and 0.8 mg of TPGS were dissolved in 2 mL of water to obtain an aqueous phase, the organic phase and the aqueous phase were quickly mixed and subjected to probe sonication at 130 W for 25 min using an ultrasonic cell disruptor in an ice bath, and then the organic solvent was removed by dialysis to obtain a suspension of Til NCs with a particle size of 104.74 nm±0.26 nm and a PDI of 0.45±0.23. The Til NCs were used as a drug-carrying core, soybean lecithin was used as a main lipid, and Chol was used as a main component, a FA-modified phospholipid bilayer was constructed on the surface of Til NCs by a combined method of thin film hydration-ultrasonic treatment to form FA-Lipo@Til NCs with a shell-core structure: the soybean lecithin, Chol, mPEG2000-Chol, and FA-PEG2000-Chol had masses of 6 mg, 1.5 mg, 0.25 mg, and 0.025 mg, respectively (FA accounted for 0.3% of the lipid content), and were dissolved in 6 mL of anhydrous ethanol, the anhydrous ethanol in the round-bottom flask was removed by rotary evaporation in a 60° C. water bath, and then the auxiliary materials were evenly spread on the bottom of the flask to form a dense lipid film by rotary evaporation for 30 min. 2 mL of suspension of Til NCs, containing 9 mg of drug (Til), was poured into the round-bottom flask forming the lipid film to allow hydration at room temperature for 30 min. The samples were subjected to ultrasonic treatment at 120 W for 10 min in an ice bath. Finally, FA-Lipo@Til NCs were obtained, with a particle size of 147.86 nm±1.55 nm, a PDI of 0.43±0.01, an encapsulation efficiency of (92.04±0.01) %, and a drug loading capacity of (49.38±0.01) %.

### Example 7

9 mg of Til was dissolved in 100 μL of an organic solvent of anhydrous ethanol: DMF (at a volume ratio of 1:1) to obtain an organic phase, 3 mg of PVA and 0.8 mg of TPGS were dissolved in 2 mL of water to obtain an aqueous phase, the organic phase and the aqueous phase were quickly mixed and subjected to probe sonication at 130 W for 25 min using an ultrasonic cell disruptor in an ice bath, and then the organic solvent was removed by dialysis to obtain a suspension of Til NCs with a particle size of 107.10 nm±0.23 nm and a PDI of 0.49±0.10. The Til NCs were used as a drug-carrying core, soybean lecithin was used as a main lipid, and Chol was used as a main component, a FA-modified phospholipid bilayer was constructed on the surface of Til NCs by a combined method of thin film hydration-ultrasonic treatment to form FA-Lipo@Til NCs with a shell-core structure: the soybean lecithin, Chol, mPEG2000-Chol, and FA-PEG2000-Chol had masses of 6 mg, 1.5 mg, 0.25 mg, and 0.05 mg, respectively (FA accounted for 0.6% of the lipid content), and were dissolved in 6 mL of anhydrous ethanol, the anhydrous ethanol in the round-bottom flask was removed by rotary evaporation in a 60° C. water bath, and then the auxiliary materials were evenly spread on the bottom of the flask to form a dense lipid film by rotary evaporation for 30 min. 2 mL of suspension of Til NCs, containing 9 mg of drug (Til), was poured into the round-bottom flask forming the lipid film to allow hydration at room temperature for 30 min. The samples were subjected to ultrasonic treatment at 120 W for 10 min in an ice bath. Finally, FA-Lipo@Til NCs were obtained, with a particle size of 139.26 nm±0.55 nm, a PDI of 0.39±0.01, an encapsulation efficiency of (92.05±0.01) %, and a drug loading capacity of (49.31±0.01) %.

### Example 8

9 mg of Til was dissolved in 100 μL of an organic solvent of anhydrous ethanol: DMF (at a volume ratio of 1:1) to obtain an organic phase, 3 mg of PVA and 0.8 mg of TPGS were dissolved in 2 mL of water to obtain an aqueous phase, the organic phase and the aqueous phase were quickly mixed and subjected to probe sonication at 130 W for 25 min using an ultrasonic cell disruptor in an ice bath, and then the organic solvent was removed by dialysis to obtain a suspension of Til NCs with a particle size of 102.10 nm±0.02 nm and a PDI of 0.43±0.01. The Til NCs were used as a drug-carrying core, soybean lecithin was used as a main lipid, and Chol was used as a main component, a FA-modified phospholipid bilayer was constructed on the surface of Til NCs by a combined method of thin film hydration-ultrasonic treatment to form FA-Lipo@Til NCs with a shell-core structure: the soybean lecithin, Chol, mPEG2000-Chol, and FA-PEG2000-Chol had masses of 6 mg, 1.5 mg, 0.25 mg, and 0.10 mg, respectively (FA accounted for 1.20% of the lipid content), and were dissolved in 6 mL of anhydrous ethanol, the anhydrous ethanol in the round-bottom flask was removed by rotary evaporation in a 60° C. water bath, and then the auxiliary materials were evenly spread on the bottom of the flask to form a dense lipid film by rotary evaporation for 30 min. 2 mL of suspension of Til NCs, containing 9 mg of drug (Til), was poured into the round-bottom flask forming the lipid film to allow hydration at room temperature for 30 min. The samples were subjected to ultrasonic treatment at 120 W for 10 min in an ice bath. Finally, FA-Lipo@Til NCs were obtained, with a particle size of 137.86 nm±1.96 nm, a PDI of 0.40±0.01, an encapsulation efficiency of (92.02±0.01) %, and a drug loading capacity of (49.15±0.01) %.

### Example 9

9 mg of Til was dissolved in 100 μL of an organic solvent of anhydrous ethanol: DMF (at a volume ratio of 1:1) to obtain an organic phase, 3 mg of PVA and 0.8 mg of TPGS were dissolved in 2 mL of water to obtain an aqueous phase, the organic phase and the aqueous phase were quickly mixed and subjected to probe sonication at 130 W for 25 min using an ultrasonic cell disruptor in an ice bath, and then the organic solvent was removed by dialysis to obtain a suspension of Til NCs with a particle size of 104.25 nm±0.65 nm and a PDI of 0.63±0.01. The Til NCs were used as a drug-carrying core, soybean lecithin was used as a main lipid, and Chol was used as a main component, a FA-modified phospholipid bilayer was constructed on the surface of Til NCs by a combined method of thin film hydration-ultrasonic treatment to form FA-Lipo@Til NCs with a shell-core structure: the soybean lecithin, Chol, mPEG2000-Chol, and FA-PEG2000-Chol had masses of 6 mg, 1.5 mg, 0.25 mg, and 0.50 mg, respectively (FA accounted for 6.00% of the lipid content), and were dissolved in 6 mL of anhydrous ethanol, the anhydrous ethanol in the round-bottom flask was removed by rotary evaporation in a 60° C. water bath, and then the auxiliary materials were evenly spread on the bottom of the flask to form a dense lipid film by rotary evaporation for 30 min. 2 mL of suspension of Til NCs, containing 9 mg of drug (Til), was poured into the round-bottom flask forming the lipid film to allow hydration at room temperature for 30 min. The samples were subjected to ultrasonic treatment at 120 W for 10 min in an ice bath. Finally, FA-Lipo@Til NCs were obtained, with a particle size of 139.73 nm±0.35 nm, a PDI of 0.35±0.01, an encapsulation efficiency of (92.03±0.02) %, and a drug loading capacity of (48.01±0.01) %.

### Example 10

9 mg of Til and 0.004 mg of coumarin 6 (Cou 6) were dissolved in 100 μL of an organic solvent of anhydrous ethanol: DMF (at a volume ratio of 1:1) to obtain an organic phase, 3 mg of PVA and 0.8 mg of TPGS were dissolved in 2 mL of water to obtain an aqueous phase, the organic phase and the aqueous phase were quickly mixed and subjected to probe sonication at 130 W for 25 min using an ultrasonic cell disruptor in an ice bath, the solution was reconstituted by centrifugation 3 times to remove free Cou 6, and then the organic solvent was removed by dialysis to obtain a suspension of Cou 6-labeled Til NCs (Til NCs/Cou 6) with a particle size of 106.83 nm±0.24 nm and a PDI of 0.43±0.38. The soybean lecithin, Chol, mPEG2000-Chol, and FA-PEG2000-Chol had masses of 6 mg, 1.5 mg, 0.25 mg, and 0.025 mg, respectively (FA accounted for 0.3% of the lipid content), and were dissolved in 6 mL of anhydrous ethanol, the anhydrous ethanol in the round-bottom flask was removed by rotary evaporation in a 60° C. water bath, and then the auxiliary materials were evenly spread on the bottom of the flask to form a dense lipid film by rotary evaporation for 30 min. 2 mL of suspension of Til NCs/Cou 6, containing 9 mg of drug (Til), was poured into the round-bottom flask forming the lipid film to allow hydration at room temperature for 30 min. The samples were subjected to ultrasonic treatment at 120 W for 10 min in an ice bath. Finally, FA-Lipo@Til NCs/Cou6 labeled with Cou6 were obtained, with a particle size of 125.56 nm±1.01 nm, a PDI of 0.47±0.03, an encapsulation efficiency of (91.04±0.03) %, and a drug loading capacity of (48.83±0.03) %.

RAW264.7 cells were inoculated in confocal glass-bottomed dishes at a concentration of 0.5×10⁵ cells/dish and cultured in DMEM containing 10% fetal bovine serum for 24 h; the cells were incubated in a medium containing LPS at a final concentration of 100 ng/ml and IFN-γ at a final concentration of 20 ng/mL for 24 h to allow cell induction; a Cou 6-labeled preparation containing 0.3% FA was added and incubated for 2 h; 3 replicate wells were set up in each group. After incubation, the old medium was removed and the cells were washed 3 times with PBS; the cells were fixated with 500 μL of fixative for 20 min and then washed 2 times with PBS; the cells were stained with 1× intracellular staining permeabilization buffer containing 1% DAPI for 10 min; the cells were washed 2 times with PBS. The cells were added with 1 mL of PBS and observed and photographed using CLSM LSM 880; a mean fluorescence intensity of macrophage uptake was 67.94±5.65.

### Example 11

9 mg of Til and 0.004 mg of Cou 6 were dissolved in 100 μL of an organic solvent of anhydrous ethanol: DMF (at a volume ratio of 1:1) to obtain an organic phase, 3 mg of PVA and 0.8 mg of TPGS were dissolved in 2 mL of water to obtain an aqueous phase, the organic phase and the aqueous phase were quickly mixed and subjected to probe sonication at 130 W for 25min using an ultrasonic cell disruptor in an ice bath, the solution was reconstituted by centrifugation 3 times to remove free Cou 6, and then the organic solvent was removed by dialysis to obtain a suspension of Cou 6-labeled Til NCs (Til NCs/Cou 6) with a particle size of 102.31 nm±0.37 nm and a PDI of 0.45±0.01. The soybean lecithin, Chol, mPEG2000-Chol, and FA-PEG2000-Chol had masses of 6 mg, 1.5 mg, 0.25 mg, and 0.05 mg, respectively (FA accounted for 0.6% of the lipid content), and were dissolved in 6 mL of anhydrous ethanol, the anhydrous ethanol in the round-bottom flask was removed by rotary evaporation in a 60° C. water bath, and then the auxiliary materials were evenly spread on the bottom of the flask to form a dense lipid film by rotary evaporation for 30 min. 2 mL of suspension of Til NCs, containing 9 mg of drug (Til), was poured into the round-bottom flask forming the lipid film to allow hydration at room temperature for 30 min. The samples were subjected to ultrasonic treatment at 120 W for 10 min in an ice bath. Finally, FA-Lipo@Til NCs/Cou6 labeled with Cou6 were obtained, with a particle size of 153.31 nm±0.37 nm, a PDI of 0.54±0.01, an encapsulation efficiency of (91.25±0.01) %, and a drug loading capacity of (48.87±0.01) %.

RAW264.7 cells were inoculated in confocal glass-bottomed dishes at a concentration of 0.5×10⁵ cells/dish and cultured in DMEM containing 10% fetal bovine serum for 24 h; the cells were incubated in a medium containing LPS at a final concentration of 100 ng/ml and IFN-γ at a final concentration of 20 ng/ml for 24 h to allow cell induction; a Cou 6-labeled preparation containing 0.6% FA was added and incubated for 2 h; 3 replicate wells were set up in each group. After incubation, the old medium was removed and the cells were washed 3 times with PBS; the cells were fixated with 500 μL of fixative for 20 min and then washed 2 times with PBS; the cells were stained with 1× intracellular staining permeabilization buffer containing 1% DAPI for 10 min; the cells were washed 2 times with PBS. The cells were added with 1 mL of PBS and observed and photographed using CLSM LSM 880; a mean fluorescence intensity of macrophage uptake was 72.97±0.90.

### Example 12

9 mg of Til and 0.004 mg of Cou 6 were dissolved in 100 μL of an organic solvent of anhydrous ethanol: DMF (at a volume ratio of 1:1) to obtain an organic phase, 3 mg of PVA and 0.8 mg of TPGS were dissolved in 2 mL of water to obtain an aqueous phase, the organic phase and the aqueous phase were quickly mixed and subjected to probe sonication at 130 W for 25 min using an ultrasonic cell disruptor in an ice bath, the solution was reconstituted by centrifugation 3 times to remove free Cou 6, and then the organic solvent was removed by dialysis to obtain a suspension of Cou 6-labeled Til NCs (Til NCs/Cou 6) with a particle size of 104.25 nm±0.02 nm and a PDI of 0.53±0.01. The soybean lecithin, Chol, mPEG2000-Chol, and FA-PEG2000-Chol had masses of 6 mg, 1.5 mg, 0.25 mg, and 0.10 mg, respectively (FA accounted for 1.20% of the lipid content), and were dissolved in 6 mL of anhydrous ethanol, the anhydrous ethanol in the round-bottom flask was removed by rotary evaporation in a 60° C. water bath, and then the auxiliary materials were evenly spread on the bottom of the flask to form a dense lipid film by rotary evaporation for 30 min. 2 mL of suspension of Til NCs, containing 9 mg of drug (Til), was poured into the round-bottom flask forming the lipid film to allow hydration at room temperature for 30 min. The samples were subjected to ultrasonic treatment at 120 W for 10 min in an ice bath. Finally, FA-Lipo@Til NCs/Cou6 labeled with Cou6 were obtained, with a particle size of 139.39 nm±1.29 nm, a PDI of 0.48±0.01, an encapsulation efficiency of (92.08±0.01) %, and a drug loading capacity of (49.13±0.01) %.

RAW264.7 cells were inoculated in confocal glass-bottomed dishes at a concentration of 0.5x105 cells/dish and cultured in DMEM containing 10% fetal bovine serum for 24 h; the cells were incubated in a medium containing LPS at a final concentration of 100 ng/ml and IFN-γ at a final concentration of 20 ng/mL for 24 h to allow cell induction; a Cou 6-labeled preparation containing 1.2% FA was added and incubated for 2 h; 3 replicate wells were set up in each group. After incubation, the old medium was removed and the cells were washed 3 times with PBS; the cells were fixated with 500 μL of fixative for 20 min and then washed 2 times with PBS; the cells were stained with 1× intracellular staining permeabilization buffer containing 1% DAPI for 10 min; the cells were washed 2 times with PBS. The cells were added with 1 mL of PBS and observed and photographed using CLSM LSM 880; a mean fluorescence intensity of macrophage uptake was 120.28±3.78.

### Example 13

9 mg of Til and 0.004 mg of Cou 6 were dissolved in 100 μL of an organic solvent of anhydrous ethanol: DMF (at a volume ratio of 1:1) to obtain an organic phase, 3 mg of PVA and 0.8 mg of TPGS were dissolved in 2 mL of water to obtain an aqueous phase, the organic phase and the aqueous phase were quickly mixed and subjected to probe sonication at 130 W for 25 min using an ultrasonic cell disruptor in an ice bath, the solution was reconstituted by centrifugation 3 times to remove free Cou 6, and then the organic solvent was removed by dialysis to obtain a suspension of Cou 6-labeled Til NCs (Til NCs/Cou 6) with a particle size of 109.08 nm±0.52 nm and a PDI of 0.29±0.01. The soybean lecithin, Chol, mPEG2000-Chol, and FA-PEG2000-Chol had masses of 6 mg, 1.5 mg, 0.25 mg, and 0.25 mg, respectively (FA accounted for 3.00% of the lipid content), and were dissolved in 6 mL of anhydrous ethanol, the anhydrous ethanol in the round-bottom flask was removed by rotary evaporation in a 60° C. water bath, and then the auxiliary materials were evenly spread on the bottom of the flask to form a dense lipid film by rotary evaporation for 30 min. 2 mL of suspension of Til NCs, containing 9 mg of drug (Til), was poured into the round-bottom flask forming the lipid film to allow hydration at room temperature for 30 min. The samples were subjected to ultrasonic treatment at 120 W for 10 min in an ice bath. Finally, FA-Lipo@Til NCs/Cou6 labeled with Cou6 were obtained, with a particle size of 140.5 nm±0.23 nm, a PDI of 0.24±0.05, an encapsulation efficiency of (92.08±0.01) %, and a drug loading capacity of (49.69±0.01) %.

RAW264.7 cells were inoculated in confocal glass-bottomed dishes at a concentration of 0.5×10⁵ cells/dish and cultured in DMEM containing 10% fetal bovine serum for 24 h; the cells were incubated in a medium containing LPS at a final concentration of 100 ng/ml and IFN-γ at a final concentration of 20 ng/mL for 24 h to allow cell induction; a Cou 6-labeled preparation containing 3.00% FA was added and incubated for 2 h; 3 replicate wells were set up in each group. After incubation, the old medium was removed and the cells were washed 3 times with PBS; the cells were fixated with 500 μL of fixative for 20 min and then washed 2 times with PBS; the cells were stained with 1× intracellular staining permeabilization buffer containing 1% DAPI for 10 min; the cells were washed 2 times with PBS. The cells were added with 1 mL of PBS and observed and photographed using CLSM LSM 880; a mean fluorescence intensity of macrophage uptake was 148.82±5.07.

### Example 14

9 mg of Til and 0.004 mg of Cou 6 were dissolved in 100 μL of an organic solvent of anhydrous ethanol: DMF (at a volume ratio of 1:1) to obtain an organic phase, 3 mg of PVA and 0.8 mg of TPGS were dissolved in 2 mL of water to obtain an aqueous phase, the organic phase and the aqueous phase were quickly mixed and subjected to probe sonication at 130 W for 25 min using an ultrasonic cell disruptor in an ice bath, the solution was reconstituted by centrifugation 3 times to remove free Cou 6, and then the organic solvent was removed by dialysis to obtain a suspension of Cou 6-labeled Til NCs (Til NCs/Cou 6) with a particle size of 106.25 nm±0.25 nm and a PDI of 0.41±0.01. The soybean lecithin, Chol, mPEG2000-Chol, and FA-PEG2000-Chol had masses of 6 mg, 1.5 mg, 0.25 mg, and 0.50 mg, respectively (FA accounted for 6.00% of the lipid content), and were dissolved in 6 mL of anhydrous ethanol, the anhydrous ethanol in the round-bottom flask was removed by rotary evaporation in a 60° C. water bath, and then the auxiliary materials were evenly spread on the bottom of the flask to form a dense lipid film by rotary evaporation for 30 min. 2 mL of suspension of Til NCs, containing 9 mg of drug (Til), was poured into the round-bottom flask forming the lipid film to allow hydration at room temperature for 30 min. The samples were subjected to ultrasonic treatment at 120 W for 10 min in an ice bath. Finally, FA-Lipo@Til NCs/Cou6 labeled with Cou6 were obtained, with a particle size of 134.38 nm±0.20 nm, a PDI of 0.42±0.01, an encapsulation efficiency of (92.08±0.02) %, and a drug loading capacity of (46.90±0.01) %.

RAW264.7 cells were inoculated in confocal glass-bottomed dishes at a concentration of 0.5×10⁵ cells/dish and cultured in DMEM containing 10% fetal bovine serum for 24 h; the cells were incubated in a medium containing LPS at a final concentration of 100 ng/ml and IFN-γ at a final concentration of 20 ng/mL for 24 h to allow cell induction; a Cou 6-labeled preparation containing 6.00% FA was added and incubated for 2 h; 3 replicate wells were set up in each group. After incubation, the old medium was removed and the cells were washed 3 times with PBS; the cells were fixated with 500 μL of fixative for 20 min and then washed 2 times with PBS; the cells were stained with 1× intracellular staining permeabilization buffer containing 1% DAPI for 10 min; the cells were washed 2 times with PBS. The cells were added with 1 mL of PBS and observed and photographed using CLSM LSM 880; a mean fluorescence intensity of macrophage uptake was 145.48±3.63.

### Example 15

9 mg of Til was dissolved in 100 μL of an organic solvent of anhydrous ethanol: DMF (at a volume ratio of 1:1) to obtain an organic phase, 3 mg of PVA and 0.8 mg of TPGS were dissolved in 2 mL of water to obtain an aqueous phase, the organic phase and the aqueous phase were quickly mixed and subjected to probe sonication at 130 W for 25 min using an ultrasonic cell disruptor in an ice bath, and then the organic solvent was removed by dialysis to obtain a suspension of Til NCs with a particle size of 103.10 nm±0.62 nm and a PDI of 0.24±0.06. The Til NCs were used as a drug-carrying core, soybean lecithin was used as a main lipid, and Chol was used as a main component, a FA-modified phospholipid bilayer was constructed on the surface of Til NCs by a combined method of thin film hydration-ultrasonic treatment to form FA-Lipo@Til NCs with a shell-core structure: the soybean lecithin, cholesterol, mPEG2000-Chol had masses of 6 mg, 1.5 mg, and 0.5 mg, respectively, and were dissolved in 6 mL of anhydrous ethanol, the anhydrous ethanol in the round-bottom flask was removed by rotary evaporation in a 60° C. water bath, and then the auxiliary materials were evenly spread on the bottom of the flask to form a dense lipid film by rotary evaporation for 30 min. 2 mL of suspension of Til NCs, containing 9 mg of drug (Til), was poured into the round-bottom flask forming the lipid film to allow hydration at room temperature for 30 min. The samples were subjected to ultrasonic treatment at 120 W for 10 min in an ice bath. Finally, Lipo@Til NCs without FA modification were obtained, with a particle size of 132.82 nm±0.03 nm, a PDI of 0.47±0.01, an encapsulation efficiency of (92.06±0.01) %, and a drug loading capacity of (48.71±0.01) %.

### Example 16

9 mg of Til and 0.4 mg of DiD were dissolved in 100 μL of an organic solvent of anhydrous ethanol: DMF (at a volume ratio of 1:1) to obtain an organic phase, 3 mg of PVA and 0.8 mg of TPGS were dissolved in 2 mL of water to obtain an aqueous phase, the organic phase and the aqueous phase were quickly mixed and subjected to probe sonication at 130 W for 25 min using an ultrasonic cell disruptor in an ice bath, the solution was reconstituted by centrifugation 3 times to remove free DiD, and then the organic solvent was removed by dialysis to obtain a suspension of DiD-labeled Til NCs (Til NCs/DiD) with a particle size of 112.26 nm±0.19 nm and a PDI of 0.49±0.04. The soybean lecithin, Chol, mPEG2000-Chol, and FA-PEG2000-Chol had masses of 6 mg, 1.5 mg, 0.25 mg, and 0.25 mg, respectively, and were dissolved in 6 mL of anhydrous ethanol, the anhydrous ethanol in the round-bottom flask was removed by rotary evaporation in a 60° C. water bath, and then the auxiliary materials were evenly spread on the bottom of the flask to form a dense lipid film by rotary evaporation for 30 min. 2 mL of suspension of Til NCs was poured into the round-bottom flask forming the lipid film to allow hydration at room temperature for 30 min. The samples were subjected to ultrasonic treatment at 120 W for 10 min in an ice bath. Finally, FA-Lipo@Til NCs/DiD labeled with DiD were obtained, with a particle size of 146.27 nm±0.01 nm, a PDI of 0.52±0.09, an encapsulation efficiency of (90.08±0.12) %, and a drug loading capacity of (46.55±0.12) %.

The following experiments were conducted on the FA-Lipo@Til NCs of Examples 1 to 16:

### (1) Physical Properties

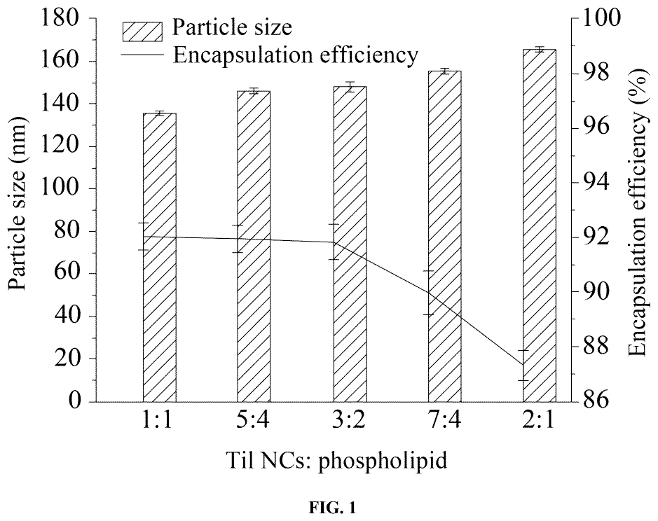

The particle size and encapsulation efficiency of FA-Lipo@Til NCs obtained in Examples 1 to 5 were tested, as shown in FIG. 1. The ratio at abscissa in FIG. 1 represented a mass ratio of Til to phospholipid during preparation, and 1:1, 5:4, 3:2, 7:4, and 2:1 in FIG. 1 corresponded to Examples 1, 2, 3, 4, and 5, respectively. The ratio of Til to phospholipid had a great influence on the particle size and encapsulation efficiency of FA-Lipo@Til NCs, and FA-Lipo@Til NCs with smaller particle size and higher encapsulation efficiency could be prepared when the mass ratio of Til to phospholipid was 3:2.

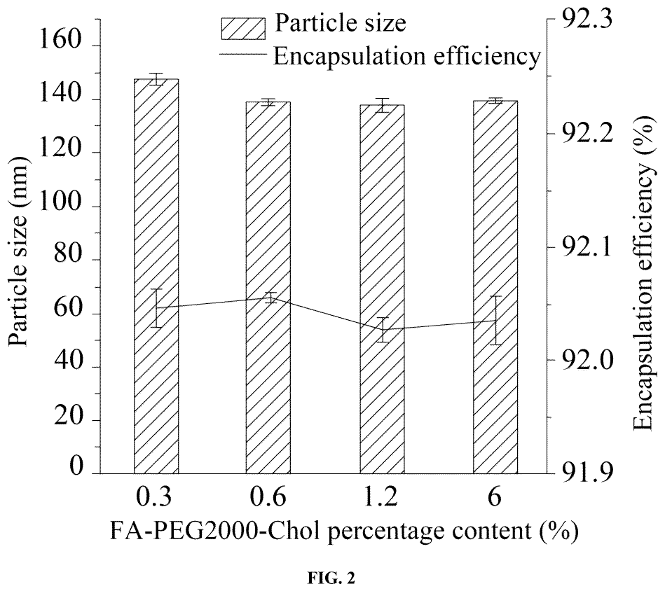

The particle size and encapsulation efficiency of the FA-Lipo@Til NCs of Examples 6 to 9 were tested, as shown in FIG. 2. The abscissa in FIG. 2 was the percentage of FA derivative in the Lipo, and 0.3%, 0.6%, 1.2%, and 6% in FIG. 2 corresponded to Examples 6, 7, 8, and 9, respectively. Different FA percentages had little influence on the particle size and encapsulation efficiency of FA-Lipo@Til NCs.

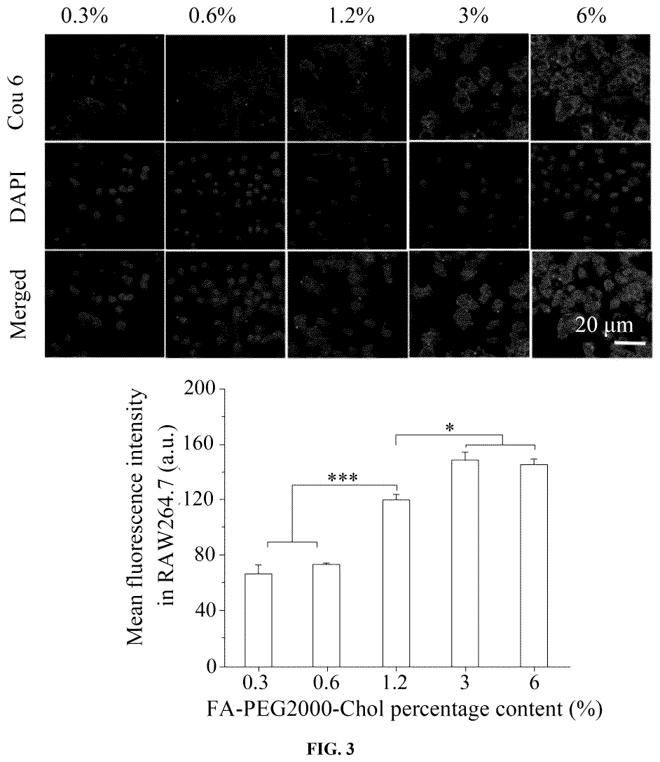

The FA-Lipo@Til NCs of Examples 10 to 14 were tested for uptake by RAW264.7, as shown in FIG. 3. In FIGS. 3, 0.3%, 0.6%, 1.2%, 3%, and 6% represented the percentage of FA derivative in Lipo, corresponding to Examples 10, 11, 12, 13, and 14, respectively; where Cou 6 represented the fluorescence intensity of FA-Lipo@Til NCs/Cou 6 taken by RAW264.7, DAPI represented the nucleus of RAW264.7 cells, and Merged represented the merging of Cou 6 and DAPI images in the same group. Preparations with different FA percentages had a greater influence on the uptake in RAW264.7, and the preparation with 3.00% FA percentage (Example 13) had a higher uptake in RAW264.7.

### (2) Appearance

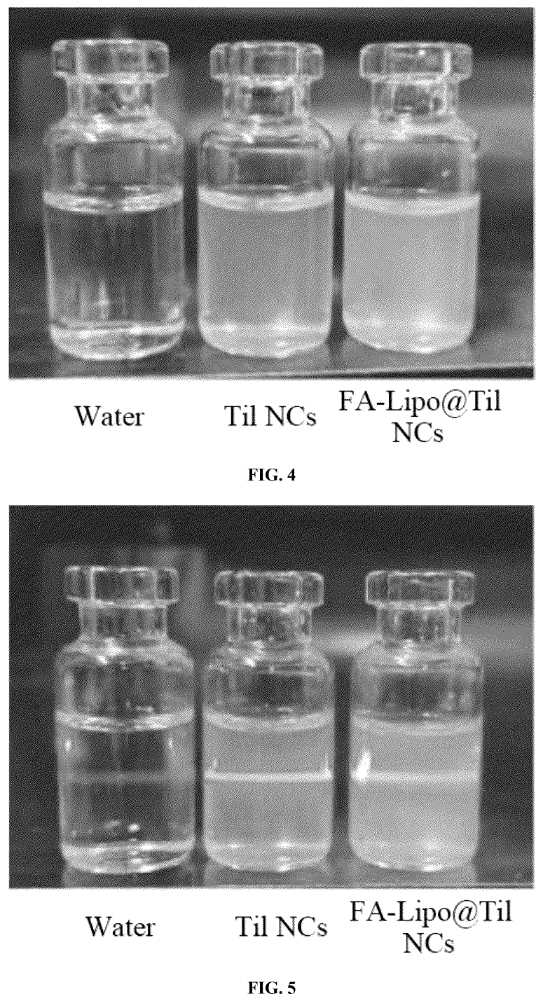

The FA-Lipo@Til NCs in Example 3 was observed with naked eye under sunlight, as shown in FIG. 4 (where FA-Lipo@Til NCs represented the folic acid-modified liposome-encapsulated tilianin nanocrystal in Example 3, and Til NCs represented the tilianin nanocrystals in Example 3), indicating that the obtained FA-Lipo@Til NCs were clear, uniform, light blue, and accompanied by opalescence.

The FA-Lipo@Til NCs and Til NCs of Example 3 were irradiated from one direction by a laser pen, as shown in FIG. 5, indicating that the FA-Lipo@Til NCs had an extremely obvious Tyndall phenomenon, indicating that FA-Lipo@Til NCs were shown in a uniform colloidal solution.

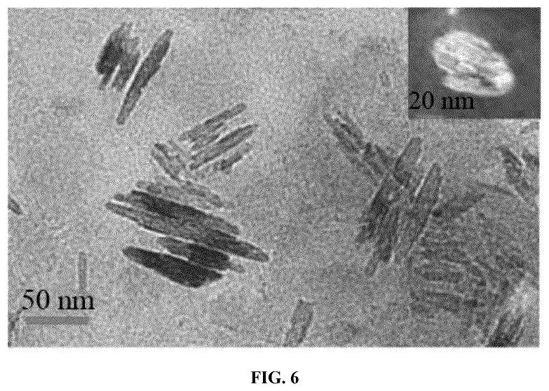

The NCs of Example 3 were observed by TEM. 5 μL of FA-Lipo@Til NCs were gently immersed on a copper grid with a carbon support film and allowed to settle at room temperature for 30 s, and then negatively stained with 2% phosphotungstic acid staining solution for 2 min. After natural drying, the copper mesh was slowly placed on an observation handle and observed by TEM, as shown in FIG. 6, indicating that the FA-Lipo@Til NCs were needle-like crystals with a core-shell structure.

### (3) Determination of Encapsulation Efficiency

The encapsulation efficiency of NCs in Example 3 was calculated according to the following Formula 1:

$$Encapsulation efficiency = ( ( total amount of drug - total amount of drug in lower supernatant ) / total amount of drug ) \times 100 \% , ; \qquad Formula 1$$

where

In Formula 1, the total amount of drug was a total mass of Til added during preparation; the total amount of drug in lower supernatant was a total mass of Til not encapsulated by Lipo membrane; the total mass of Til was determined by HPLC, and the total amount of drug in lower supernatant was determined by separating the Til encapsulated in FA-Lipo@Til NC via high-speed centrifugation, re-dissolving the drug in the lower supernatant with methanol, and then determining by HPLC.

HPLC was conducted using a Reliasil C18 column (4.6 mm×250 mm, 5 μm) with a detection wavelength of 323 nm. The encapsulation efficiency of the preparation was calculated as (91.82±0.65) %.

### (4) Determination of Drug Loading Capacity

The drug loading capacity of NCs in Example 3 was calculated according to the following Formula 2:

$$Drug loading capacity = ( ( total amount of drug - total amount of drug in lower supernatant ) / total mass of raw materials ) \times 100 \% , ; \qquad Formula 2$$

where

In Formula 2, the total mass of raw materials was a total mass of Til, phospholipid, Chol, mPEG-Chol, and FA derivative added during preparation; the total amount of drug was a total mass of Til added during preparation; the total amount of drug in lower supernatant was a total mass of Til not encapsulated by Lipo membrane; the total mass of Til was determined by HPLC, and the total amount of drug in lower supernatant was determined by separating the Til encapsulated in FA-Lipo@Til NC via high-speed centrifugation, re-dissolving the drug in the lower supernatant with methanol, and then determining by HPLC.

HPLC was conducted using a Reliasil C18 column (4.6 mm×250 mm, 5 μm) with a detection wavelength of 323 nm. The drug loading capacity of the preparation was calculated as (48.61±0.65) %.

### (5) Stability

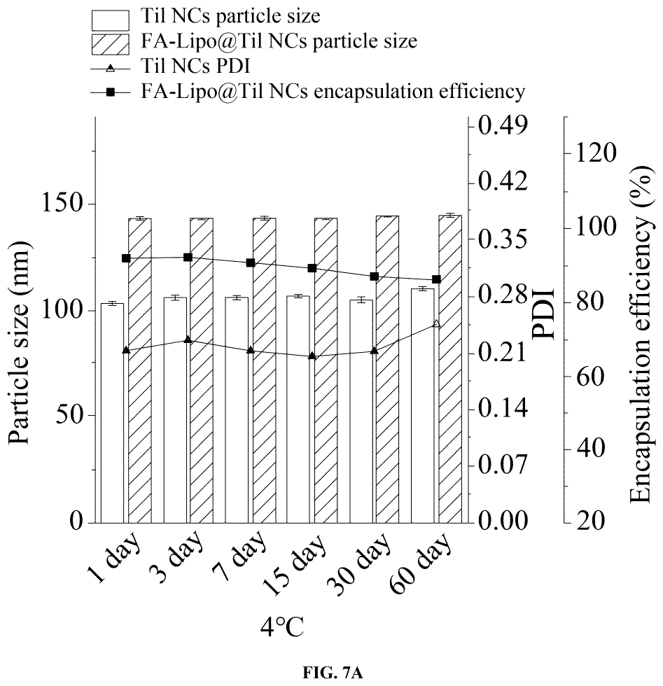

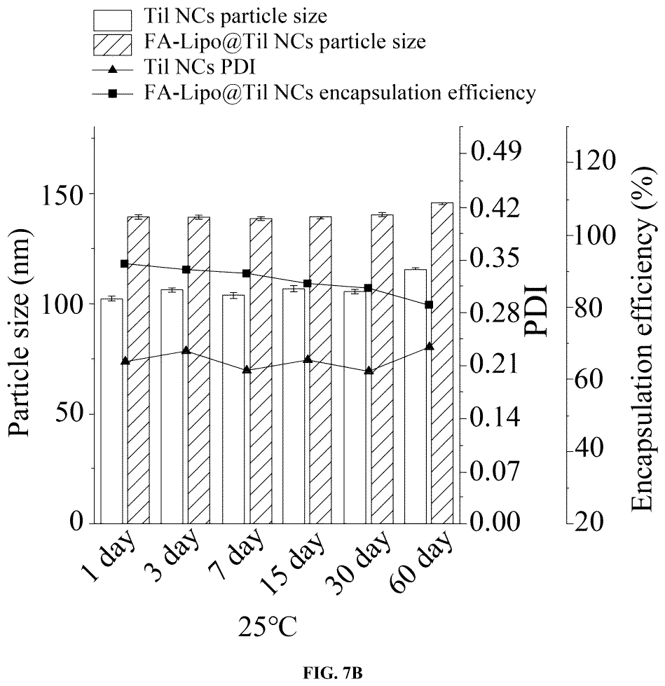

2 mL of FA-Lipo@Til NCs prepared in Example 3 were put into an EP tube, and then placed at 25° C. and 4° C. for 2 months, respectively, to observe the changes in properties, particle size, and encapsulation efficiency thereof. FIG. 7A-FIG. 7B show that the particle size of FA-Lipo@Til NCs did not change significantly within 60 days at 25° C. and 4° C. Although the encapsulation efficiency of FA-Lipo@Til NCs decreased slightly after 15 days, it was still above 80%, indicating high stability.

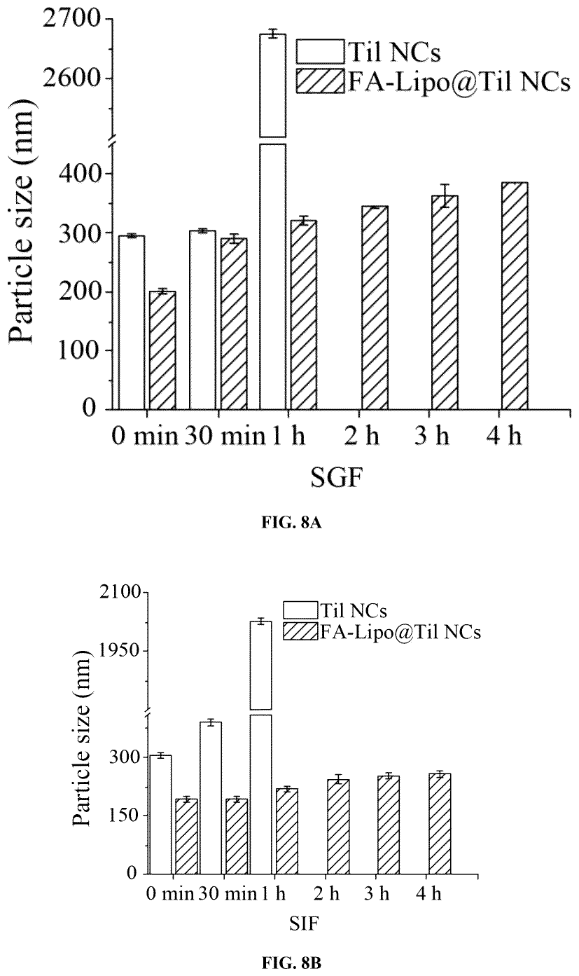

2 mL of FA-Lipo@Til NCs were added into the vial, and 6 mL of simulated gastric fluid (SGF) or simulated intestinal fluid (SIF) was added to allow mixing, an obtained mixture was placed in a constant-temperature water bath shaker for incubation under shaking (37° C., 100 rpm), and particle size of the preparation was measured at 0, 0.5, 1, 2, 3 and 4 h, while changes were recorded. FIG. 8A-FIG. 8B show that the particle size of Til NCs in SGF and SIF was in a gradual increasing trend, while the particle size of FA-Lipo@Til NCs in SIF and SGF remained basically stable within 4 h, with only a slight increase, indicating that the overall structure of FA-Lipo@Til NCs could exist stably in the intestinal fluid, thus increasing the stability of Til NCs in SGF and SIF.

(6) Dissolution behavior

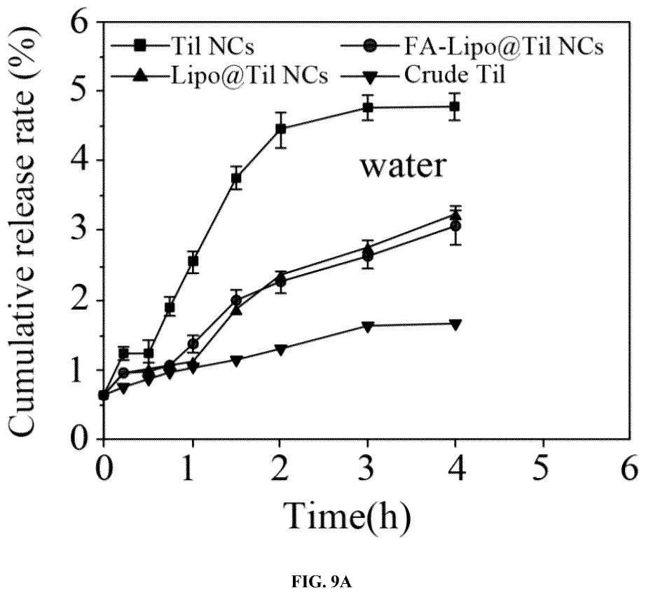

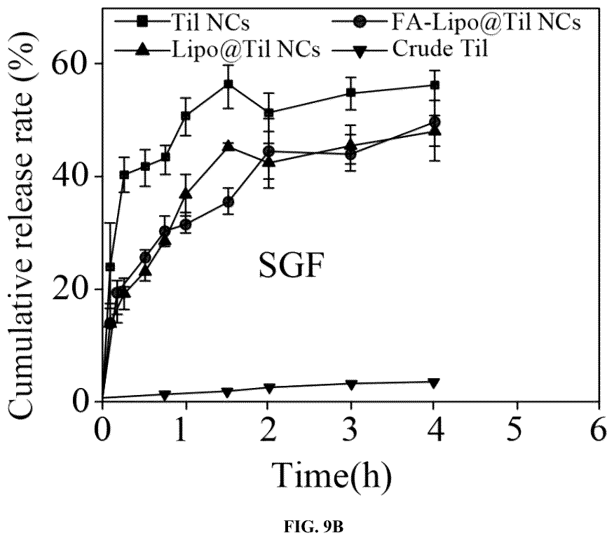

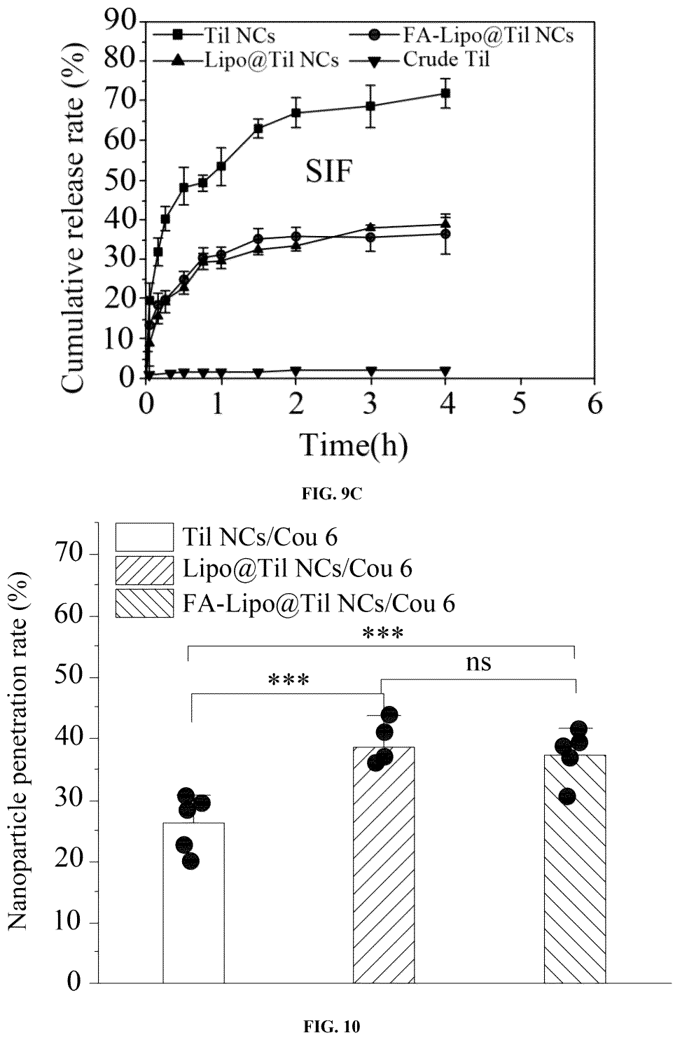

The dissolution behavior of FA-Lipo@Til NCs in Example 3 was measured in different media (water, SGF, and SIF) at 37° C. using the dialysis diffusion method. 1 mL of Til raw material and Til NC preparation (containing 1 mg/mL of Til) were placed in a dialysis bag with a molecular weight cut-off of 3,500 D, which was then placed in 50 mL of dissolution medium and then placed in an oscillator with a frequency of 100 rpm at 37° C. Samples were taken at 1 min, 2 min, 3 min, 5 min, 10 min, 15 min, 30 min, 45 min, 60 min, 90 min, 120 min, 180 min, and 240 min, respectively. After taking the samples, the corresponding volume of medium was supplemented, and the samples were centrifuged (12,000 rpm, 10 min) and determined by HPLC. FIG. 9A-FIG. 9C (in FIG. 9A-FIG. 9C, FA-Lipo@Til NCs represented folic acid-modified liposome-encapsulated tilianin nanocrystals, Crude Til represented the raw material Til, Til NCs represented tilianin nanocrystals,and Lipo@Til NCs represented tilianin-modified liposome nanocrystals prepared in Example 15) showed that the release amount of raw material Til in SGF and SIF was only about 4%. FA-Lipo@Til NCs were released more slowly than Til NCs. At 4 h, approximately 45% (in SGF) and 35% (in SIF) of Til in Lipo@Til NCs and FA-Lipo@Til NCs were released, while the cumulative release from Til NCs exceeded 55% (in SGF) and 65% (in SIF) within 4 h. Til NCs could significantly improve the solubility of Til, and the slow release of Til from Lipo@Til NCs and FA-Lipo@Til NCs encapsulated with phospholipid bilayer could be effectively controlled.

### (7) Mucus Penetration Effect

The FA-Lipo@Til NCs obtained in Example 13 were used to evaluate the mucus penetration ability of different nanoparticles using agarose gel. Agarose powder was dissolved in deionized water and heated to 100° C. in a microwave oven to obtain a 0.3% (w/v) solution, 1 mL of agarose solution was dispensed into vials. After the solution hardened, 1 mL of 20 mg/mL mucin solution was added. FA-Lipo@Til NCs/Cou 6, Til NCs/Cou 6, and the corresponding Lipo@Til NCs/Cou 6 that were labeled with Cou 6 in Example 13 were placed on the mucus layer and incubated at 37° C. in a shaker (100 r) for 6 h. The mucus layer was removed, and the agarose gel was washed, dissolved, and then detected using a 466 nm microplate reader (Thermo Scientific, USA). FIG. 10 shows that the penetration efficiency of FA-Lipo@Til NCs in the mucus layer (38.62±1.8%) was significantly higher than that of Til NCs (26.16±4.6%), indicating that phospholipid encapsulation could improve the penetration of FA-Lipo@Til NCs in the agarose gel layer.

The ability of FA-Lipo@Til NCs in Example 13 to penetrate the mucus layer was studied using a 24-well Transwell plate. The pre-made mucus (20 mg/mL) was evenly applied to the upper layer of the Transwell plate, and HBSS-balanced water was added to the lower layer and balanced at 37° C. for 30 min. 100 μL of Cou 6-labeled FA-Lipo@Til NCs were added, samples were taken from the BL side at specific time points, Cou 6 concentration was detected by microplate reader, and the apparent permeability coefficient (Papp) of each group of preparations was calculated:

$$Papp = 1 / ( A \times {C}_{0} ) \times dQ / dt \times 100 \% , ; \qquad Formula 3$$

where

In Formula 3: the cumulative amount of the preparation transported from the upper layer of the Transwell plate to the lower chamber was dQ/dt, its initial concentration on the upper side was Co, and the membrane area was A (cm²).

FIG. 11A-FIG. 11B show that the permeability of FA-Lipo@Til NCs (6.09±0.8×10⁻⁶ cm/s) was significantly higher than that of Til NCs (4.66±0.8×10⁻⁶ cm/s). Til NCs had the lowest permeability (4.66±0.8×10⁻⁶ cm/s). This indicated that compared with Til NCs not encapsulated with phospholipid, FA-Lipo@Til NCs had better mucus penetration ability.

### (8) Cell Uptake

In the present disclosure, the Caco-2 cells used were from the Cell Resource Bank of the Chinese Academy of Sciences (purchased from Shanghai), the flow cytometer used was NovoCyte™ (ACEA Biosciences), the flow cytometric analysis data were analyzed using Flo W Jo version 7.2.5 software, the CLSM used was Germany ZEISS, LSM880, and the CLSM analysis data were analyzed using Image J software.

Since FA-Lipo@Til NCs did not have fluorescent properties, in order to characterize its cellular uptake, Cou 6 was loaded into FA-Lipo@Til NCs using a co-precipitation method, as described in Example 13.

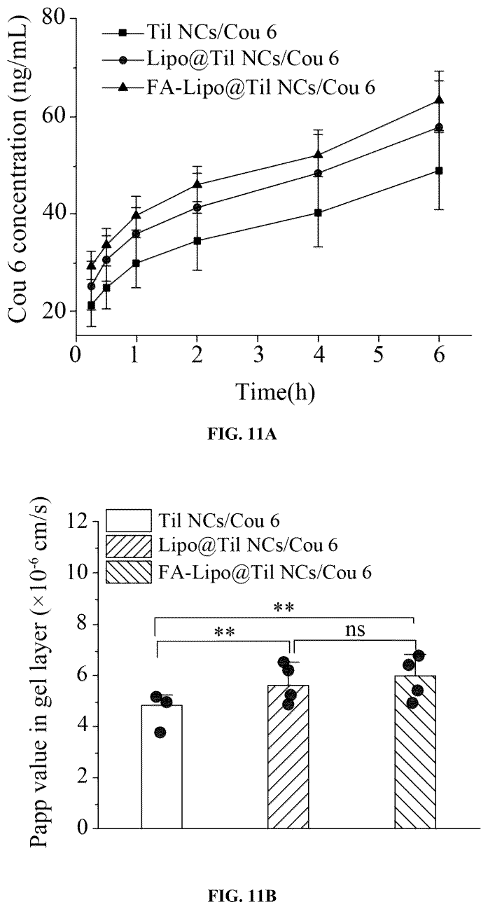

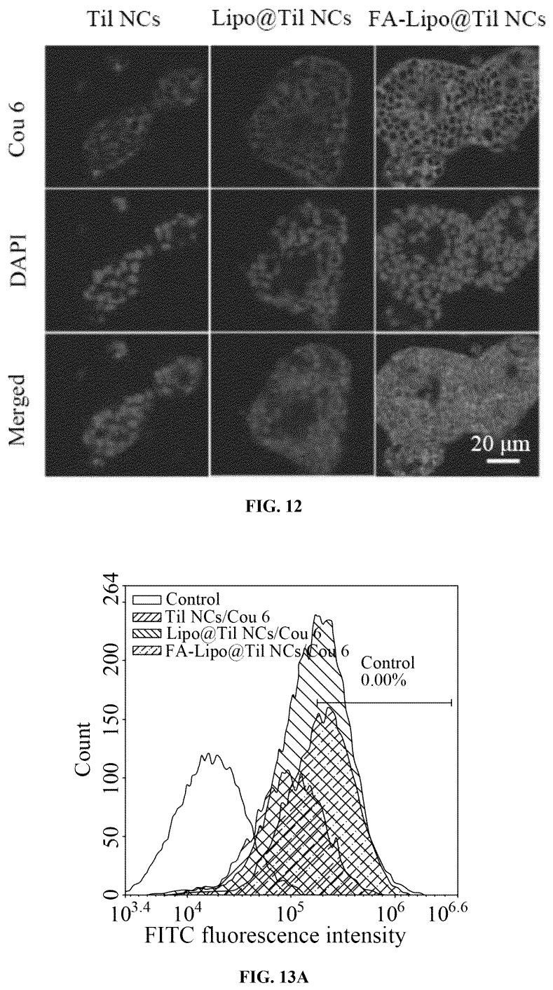

Caco-2 cells were inoculated in a 24-well plate at a density of 1×10⁵ cells/well and cultured in an incubator for 2 days. After replacing a new serum-free medium, the NCs of Example 13 were added into each well, and the uptake was terminated at 2 h. The cells were washed 2 times, and DAPI was added for staining for 10 min. After washing 2 times, the cells were observed under CLSM. FIG. 12 shows that the uptake of FA-Lipo@Til NCs byCaco-2 cells gradually increased with time.

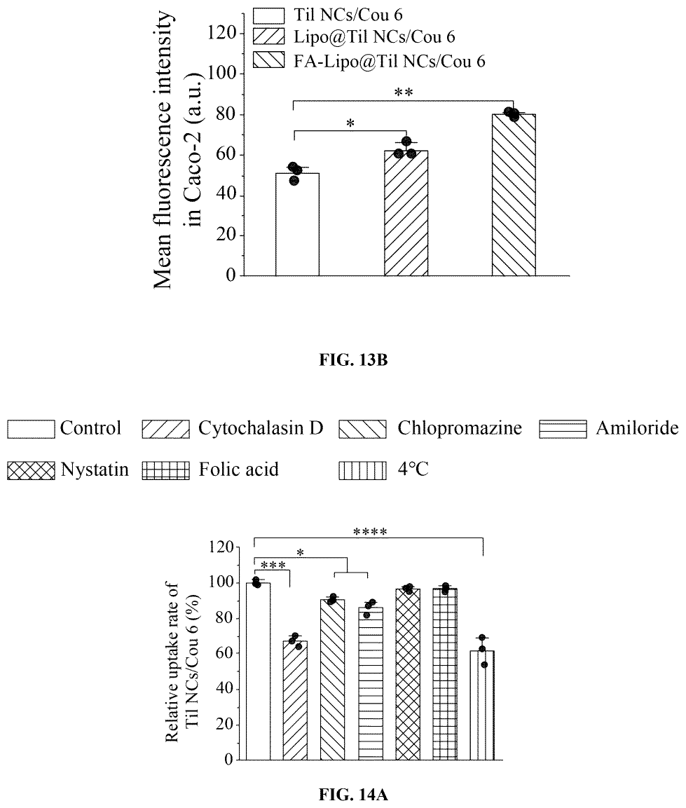

Caco-2 cells were inoculated in a 24-well plate at a density of 1×10⁵ cells/well and cultured in an incubator for 2 days. After replacing a new serum-free medium, the NCs of Example 13 were added into each well, and the uptake was terminated at 2 h. The cells were collected into a flow tube and detected by flow cytometry. FIG. 13A-FIG. 13B shows that the uptake of FA-Lipo@Til NCs by Caco-2 cells gradually increased with time.

### (9) Cell Uptake Mechanism

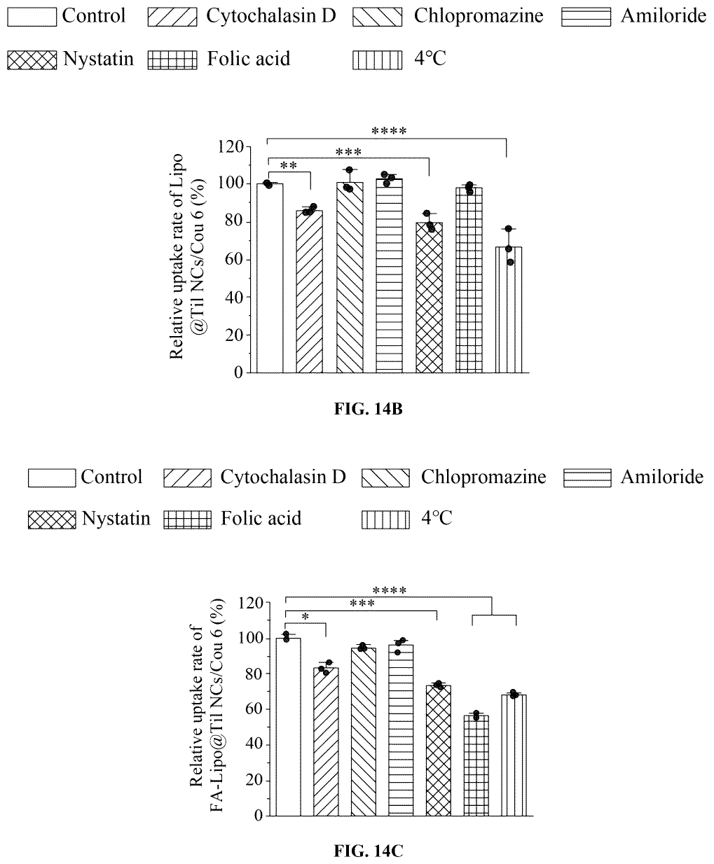

The Cou 6-labeled FA-Lipo@Til NCs of Example 13 were taken, the cells were inoculated into a 24-well plate at a density of 1×10⁵ Caco-2 cells/well, incubated for 2 days, then the original MEM medium in the 24-well plate was removed, and then actin-mediated inhibitor cytochalasin D, clathrin-mediated inhibitor chlorpromazine, macropinocytosis-mediated inhibitor amiloride, caveolin-mediated inhibitor nystatin, and FR inhibitor FA were added at final concentrations of 1 μg/mL, 20 μg/mL, 10 μg/mL, 10 μg/mL and 20 μg/mL, respectively. After 30 min of culture, FA-Lipo@Til NCs were added and incubated for 2 h, where energy inhibition group was placed in a 4° C. refrigerator away from light, and the group without inhibitor served as a positive control. After 2 h, the medium was removed, the cells were collected into flow cytometry tubes, centrifuged repeatedly, washed and prepared into cell samples, and the fluorescence intensity of Cou 6 was measured by FCM. As shown in FIG. 14A-FIG. 14C, the FA-Lipo@Til NCs had endocytosis mediated by caveolin, actin, and specific FA receptors. The cellular uptake of Til NCs involved multiple pathways including actin-and clathrin-mediated endocytosis and macropinocytosis. It was demonstrated that lipid membrane encapsulation changed the original intracellular pathway of Til NCs, and modification of FA ligands increased the specific endocytosis of FA-Lipo@Til NCs.

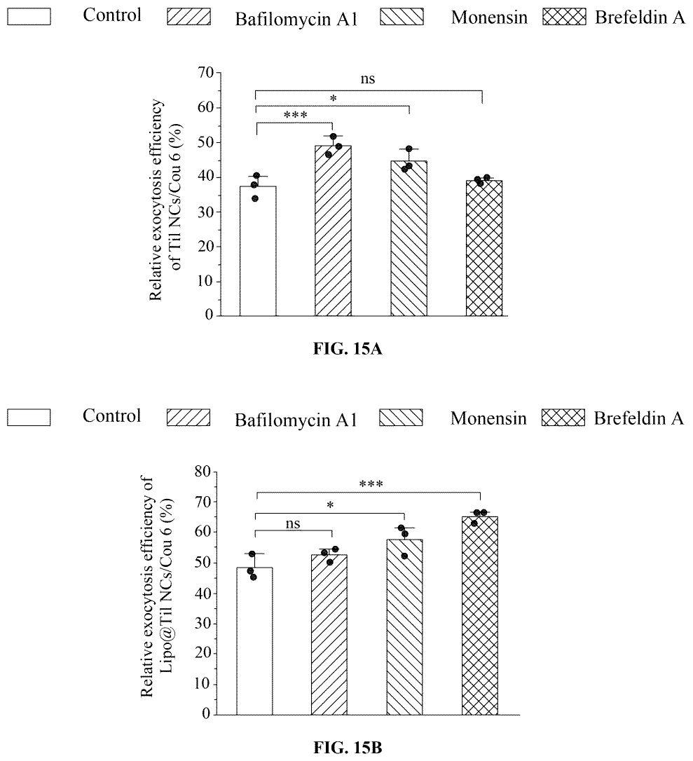

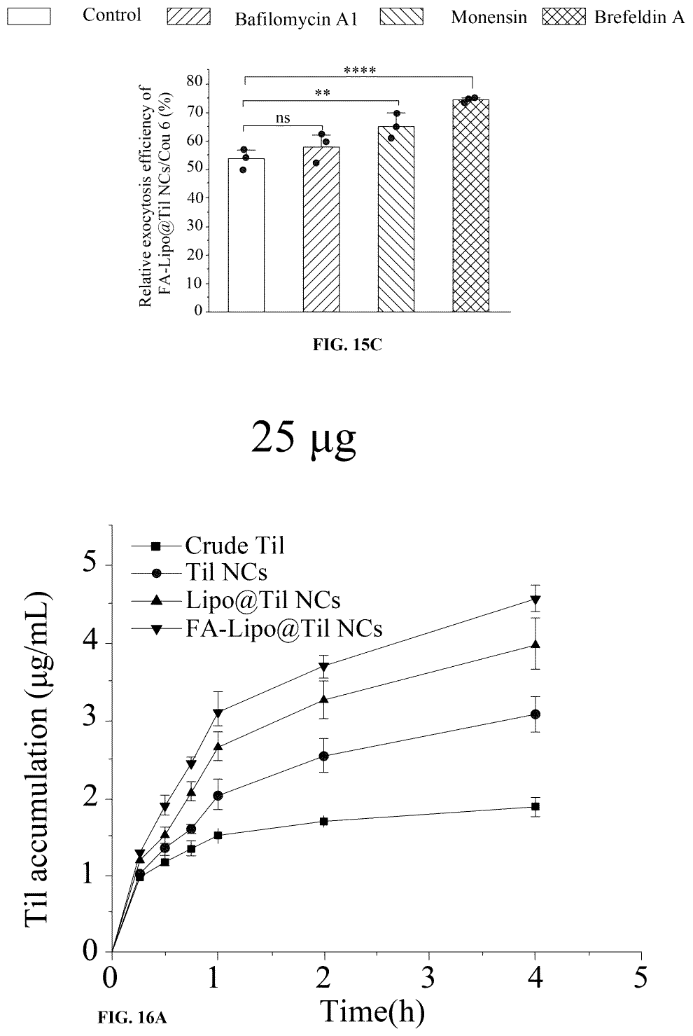

The Cou 6-labeled FA-Lipo@Til NCs of Example 13 were taken, and Caco-2 cells were inoculated in a 24-well plate at a density of 1×10⁵ cells/well and incubated for 2 days. In order to make Caco-2 cells take up more Cou 6-labeled FA-Lipo@Til NCs, the cells were incubated with Cou 6-labeled FA-Lipo@Til NCs for 4 h, and the old suspension was replaced with a medium containing lysosomal inhibitor Bafilomycin A1 (62.28 μg/mL), Golgi inhibitor Monensin (32.5 μg/mL), and inhibitor Brefeldiin A (25 μg/mL) that inhibited the transport pathway from the endoplasmic reticulum to the Golgi, and the culture was continued for 2 h. In addition, a test group without any inhibitor was used as a positive control group. After 2 h, the medium in the 24-well plate was removed and the cells were washed with 1 mL of PBS. After removing the supernatant, the cells were collected into flow cytometry tubes, washed by centrifugation, and prepared for sampling. Finally, the fluorescence intensity of Cou 6 was measured by FCM. FIG. 15A-FIG. 15C show that for Til NCs, activation of translocation after entry into cells was more common; while for FA-Lipo@Til NCs, translocation from the ER to the Golgi complex was more common.

### (10) Monolayer Transport Function

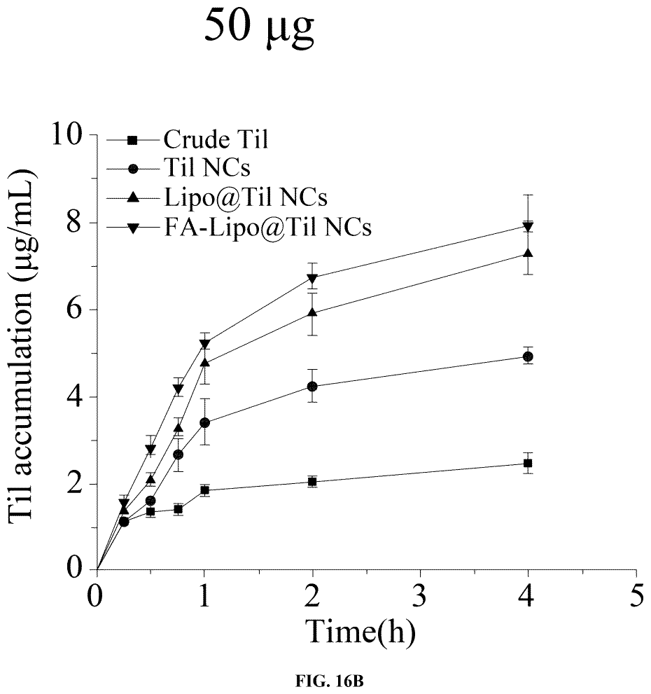

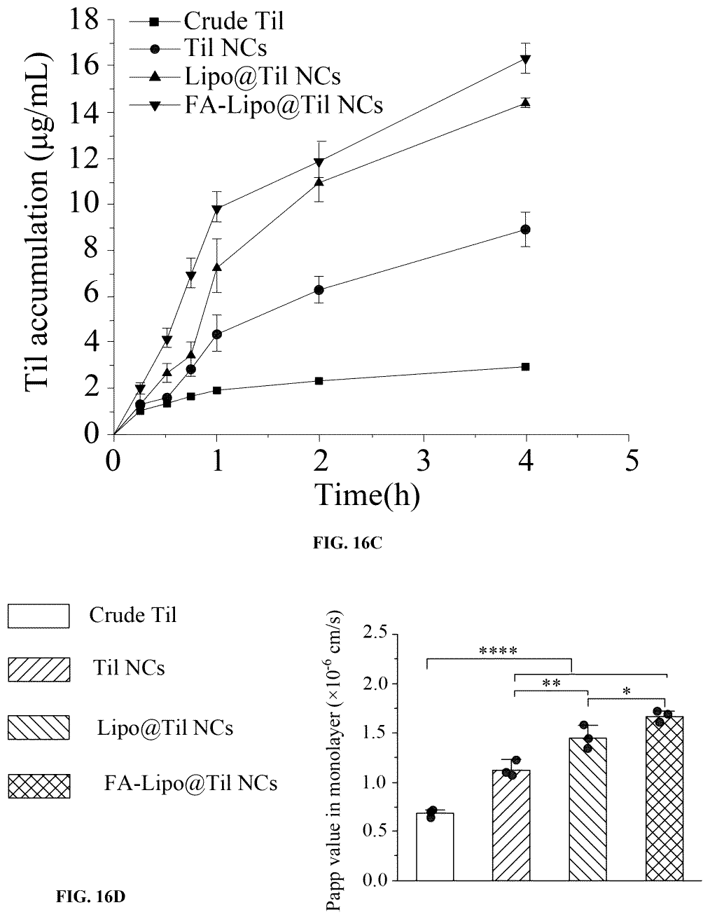

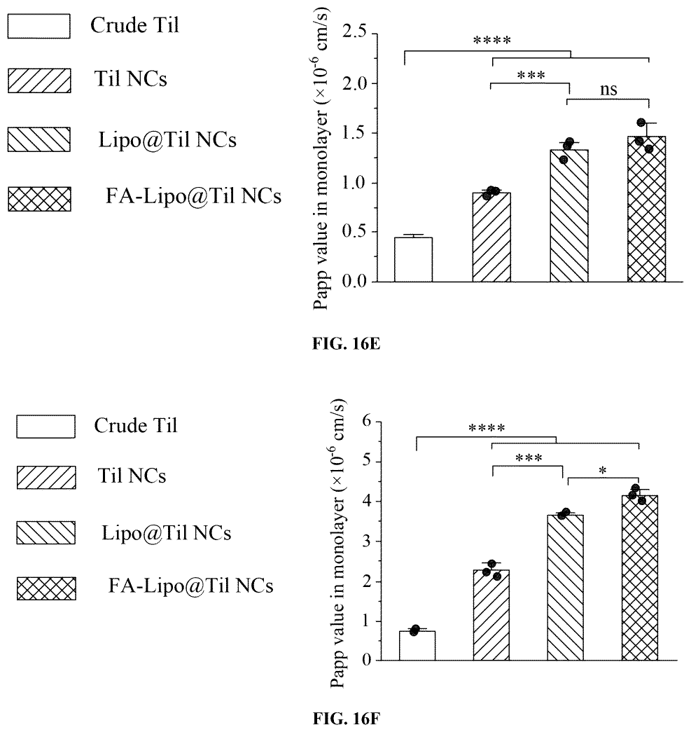

The FA-Lipo@Til NCs obtained in Example 3 were tested using a Caco-2 cell culture monolayer model, Caco-2 cells were collected with a cell plating density of 1×10⁵ cells/cm³. Cells were inoculated on the upper layer of the Transwell plate, 500 μL of diluted cell suspension was added to each well, and 600 μL of blank MEM medium was added to the lower layer, and the plates were placed in an incubator for culture; after 21 days of continuous culture, appropriate cell wells were selected, the upper and lower layers of the Transwell plate were replaced with HBSS balancing solution, and the plates were balanced in the incubator for 30 min. After removing the buffer, different concentrations of FA-Lipo@Til NCs (all diluted to 300 μL with HBSS containing 10% FBS) were added into the well, 100 μL of sample was taken from the lower layer of the Transwell plate at a specific time point, and an equal amount of HBSS buffer was supplemented, and the samples collected at different time points were used to detect the content of Til by HPLC. The cumulative release amount and Papp of the preparation were calculated. FIG. 16A-FIG. 16F show that as the dose of Til in the preparation increased from 50μg to 100 μg, the cumulative amount of Til was concentration-dependent and gradually increased with the increase in concentration. The transport amount of raw material Til through the monolayer was the lowest, while Til NCs were transported to the basolateral side over time, which was about 3 times that of the former at 4 h. The lipid bilayer coating on the surface of FA-Lipo@Til NCs increased the affinity between the nanoparticles and the cell membrane, showing excellent transport ability through the cell monolayer. FA ligands enhanced the internalization and transport of nanoparticles.

### (11) Targeting Macrophages

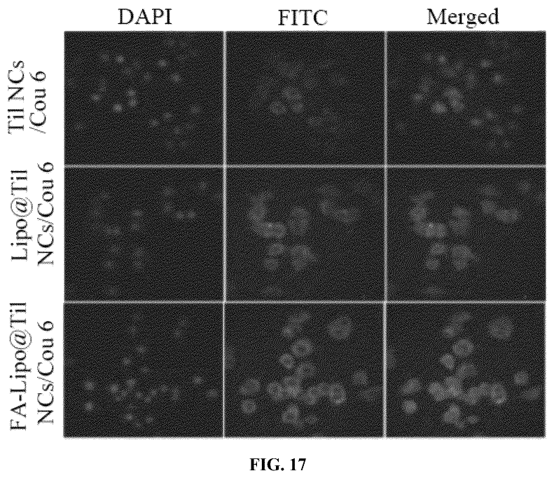

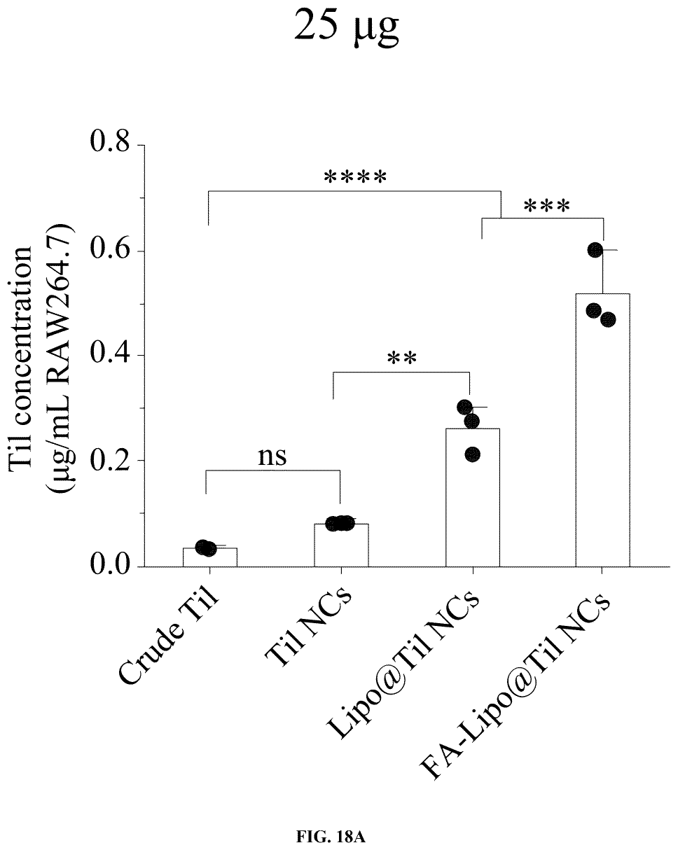

The Cou 6-labeled FA-Lipo@Til NCs of Example 13 were tested in a Caco-2/RAW264.7 co-culture model. Caco-2 cells were collected and resuspended in medium, and then counted and inoculated in the upper layer of a Transwell plate at a density of 1×10⁵ cells/cm3, with 0.5 mL per well. 1 mL of blank medium was added to the lower layer and then placed in the incubator for culture. After 21 days of co-culture, the cells were cultured as described above and RAW264.7 cells were plated in the lower chamber of Transwell, where CLSM was conducted to observe the cell crawling used in the experimental plating of the preparation targeting RAW264.7 cells across Caco-2 cells. The cells were inoculated into 24-well plates at 1×10⁵ cells/well and cultured for 24 h to allow them to adhere. RAW264.7 cells were induced by adding 100 ng/mL LPS and 20 ng/mL IFN-γ to the culture system. After incubation for 24 h, subsequent experiments were conducted. The Caco-2/RAW264.7 cell model was established. After incubation with Cou 6-labeled FA-Lipo@Til NCs for 4 h, 1× intracellular staining permeabilization buffer containing 1% DAPI was added to the lower side for staining for 10 min; the cells were washed 2 times with PBS. The slide with RAW264.7 cells on the lower side was placed upside down on a glass slide with anti-fluorescence quenching mounting medium dropped in advance, and observed and photographed using CLSM. In FIG. 17, FITC represented the fluorescence intensity of RAW264.7 uptake of FA-Lipo@Til NCs/Cou 6, DAPI represented the nucleus of RAW264.7 cells, and Merged represented the merging of FITC and DAPI images in the same group. The results showed that after the Caco-2/RAW264.7 cell model was pretreated with FA-Lipo@Til NCs for 4 h, the fluorescence intensity was enhanced compared with other preparations, indicating that FA-Lipo@Til NCs could promote the uptake of Til by macrophages in the Caco-2/RAW264.7 model.

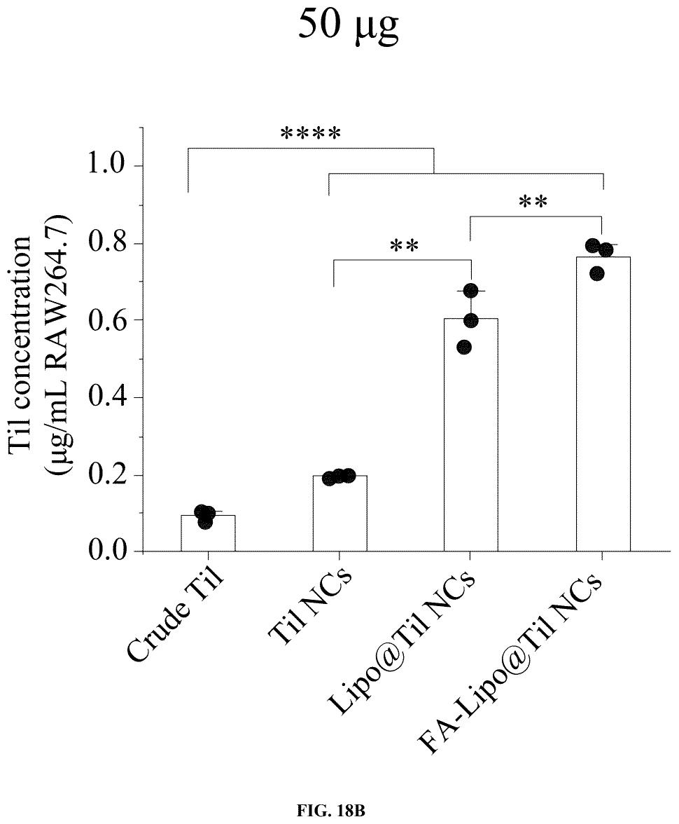

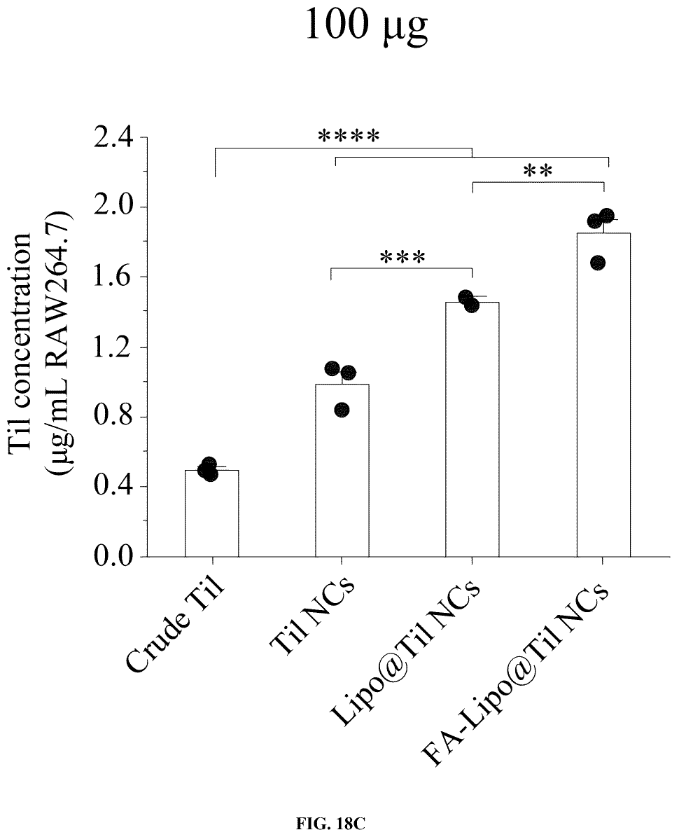

The Cou 6-labeled FA-Lipo@Til NCs of Example 13 were used as a model for Caco-2/RAW264.7 cells co-cultured in a Transwell plate. 100, 50, and 25 μg/mL of FA-Lipo@Til NCs were given and incubated for 4 h. When collecting the samples, the medium on the upper and lower sides was discarded first, and 300 μL of pure water was added and immediately mixed with a pipette tip and blown, and then transferred to a 2 mL centrifuge tube and placed on an ice box. After all the cells were transferred out, an ultrasonic cell crusher was used to disrupt the RAW264.7 cells. Each sample was ultrasonically treated for 1 min. After all the samples were disrupted, the samples of different concentrations were collected and the content of Til in RAW264.7 cells was detected by HPLC-liquid chromatography-tandem mass spectrometry (LC-MS/MS) according to the above method. FIG. 18A-FIG. 18C show that after FA-Lipo@Til NCs were incubated with the Caco-2/RAW264.7 cell model for 4 h, the accumulation of Til in macrophages was significantly increased on the original basis.

### (12) In Vivo Distribution Study

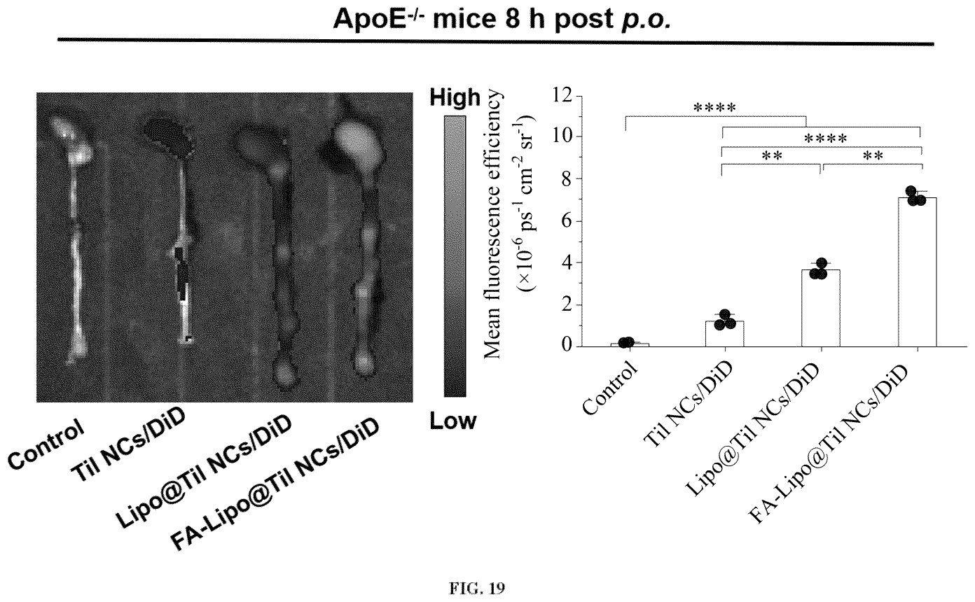

The DiD-labeled FA-Lipo@Til NCs of Example 16 were selected to feed apolipoprotein E knockout (ApoE^{-/-}) mice with a high-fat diet (purchased from Beijing HFK Bioscience Co., Ltd., Item No. H10540) for 12 weeks, and then DiD-labeled FA-Lipo@Til NCs were orally administered by gavage. Mice were sacrificed 8 h later and perfused with PBS to remove free staining; their aorta was analyzed using IVIS spectrometer. FIG. 19 shows that FA-Lipo@Til NCs accumulated more in atherosclerotic plaques such as the aortic root, aortic arch, and abdominal aorta.

### (13) Pharmacokinetic Evaluation

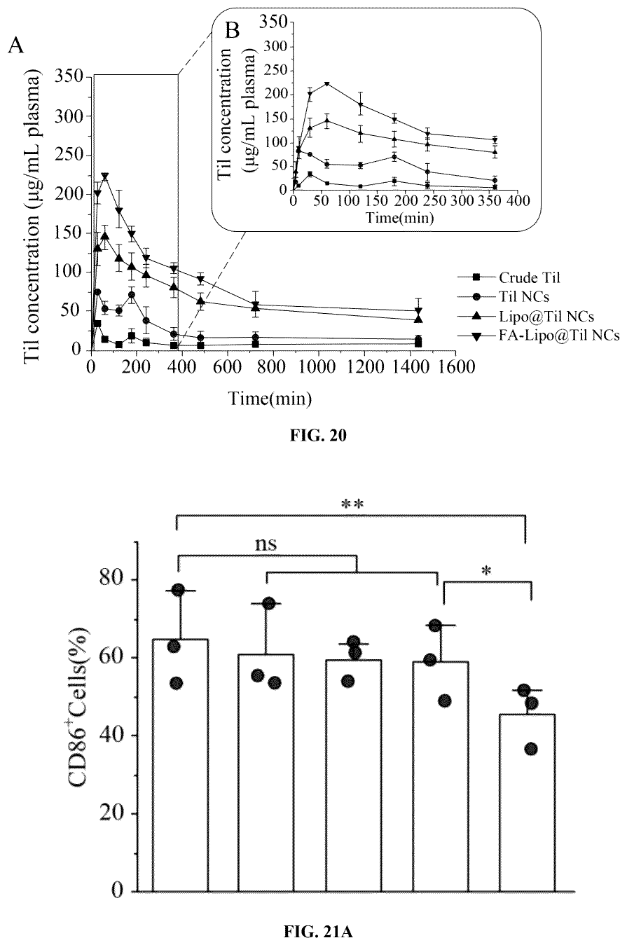

The FA-Lipo@Til NCs of Example 3 were taken, and then 30 SD rats were taken and randomly divided into 4 groups, 4 rats in each group, with free access to water. After the rats adapted to the environment for one week, they were fasted but allowed to drink water 12 h before the experiment, and were given the above 4 intragastric solutions at a dose of 20 mg/kg (based on Til), and about 0.3 mL of blood was collected from their eye sockets with a glass capillary tube at 0.083, 0.167, 0.25, 0.5, 1, 2, 3, 4, 6, 8, 12, and 24 h, and then placed in a 1.5 mL EP tube containing 10 μL of sodium heparin, mixed well and quickly centrifuged for 10 min (10,000 rpm/min), and the obtained upper plasma was transferred to another blank centrifuge tube and frozen at −20° C. LC-MS/MS was conducted to detect the content of Til in plasma at different time points. FIG. 20 shows that FA-Lipo@Til NCs had the highest Cₘₐₓ and AUC_{0-24 h}. The Cₘₐₓ was 221.60±0.65 ng/mL, which was 6.58 times, 2.94 times, and 1.53 times that of Til original drug, Til NCs, and Lipo@Til NCs, respectively, and the AUCₒ₋ₜ was 2014.25±255.35 ng/ml*h, which was 9.43 times, 3.63 times, and 1.29 times that of Til original drug, Til NCs, and Lipo@Til NCs, respectively. This suggested that FA-Lipo@Til NCs could significantly further increase the amount of nanoparticles entering the circulatory system.

### (14) Analysis of Macrophage Polarization Phenotype

Macrophages can be mainly divided into pro-inflammatory M1 type and anti-inflammatory M2 type. The CD86 receptor is highly expressed on the surface of M1 macrophages and can be used as a detection marker, while the CD206 receptor is highly expressed on the surface of M2 macrophages and can be used as a detection marker.

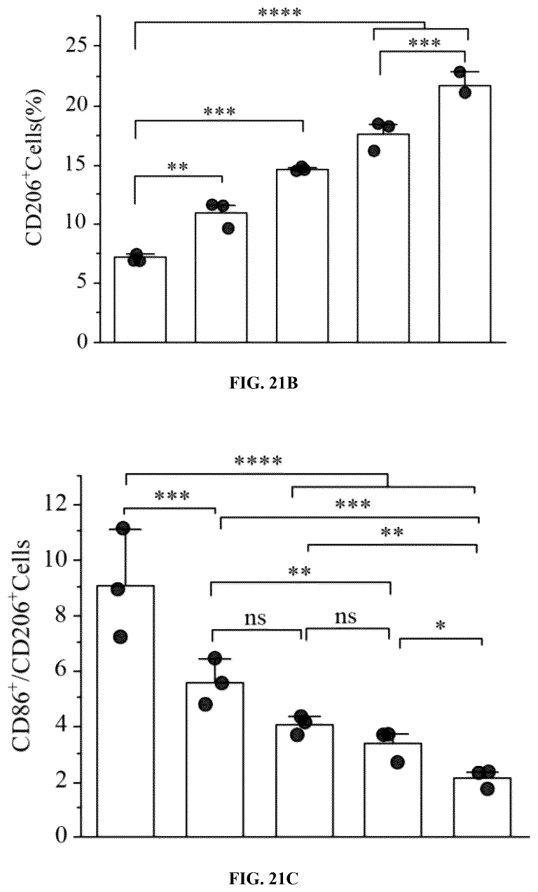

Flow cytometry was conducted to detect the polarization effect of FA-Lipo@Til NCs on RA W264.7 macrophages. The cells were inoculated in 24-well plate at a density of 1×10⁵ cells/well, with 3 wells in each group. Lipopolysaccharide (LPS) (100 ng/mL) and interferon (IFN)-γ (20 ng/ml) were added to the incubator and incubated for 24 h, then the old medium was discarded and FA-Lipo@Til NCs in Example 3 were added. After incubation for 12 h, the cells were gently pipetted into a flow tube, washed with PBS, and stained with the following antibodies in sequence for 30 min in the dark at 4° C.: FITC-labeled anti-mouse F4/80 antibody (11-4801-81, eBioscience), PE-labeled anti-mouse CD86 (17-7061-80, eBioscience), and APC-labeled anti-mouse CD206 (105007, Biolegend). After incubation, cells were rinsed and resuspended. The percentages of CD86 and CD206 were detected by flow cytometry and then analyzed using Flo W Jo software. FIG. 21A-FIG. 21C show that the proportion of CD86 positive cells in the Model group was 64.59±12.05%, and the proportion of CD206 positive cells was 7.14±0.23%. These results suggested that LPS and IFN-γ could promote the polarization of macrophages to Ml type. Although there was no significant difference between the M1 macrophages marked by CD86 and the Model group after treatment with other drug-treated groups, FA-Lipo@Til NCs could significantly inhibit the expression of CD86. In addition, FA-Lipo@Til NCs significantly promoted the increase in the mean fluorescence intensity of CD206, a marker of M2 macrophages, compared with Til NCs. Similarly, in the M1 (CD86±)/M2 (CD206±) ratio diagram, compared with the model group, the M1/M2 ratio of FA-Lipo@Til NCs decreased successively, indicating that anti-inflammatory M2 macrophages gradually dominated in this polarization system.

### (15) Analysis of Macrophage Efferocytosis

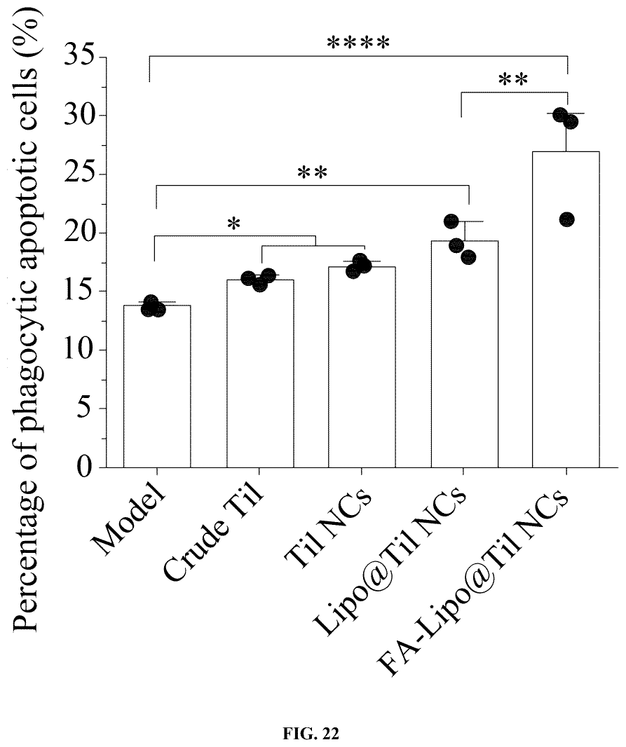

RAW264.7 cells were inoculated in 24-well plate at 1x105 cells/mL and incubated for 24 h. Induction and labeling of apoptotic cells: after the cells adhered to the well plate, the prepared cyclosporin (final concentration was 2 μM) was added to allow culturing for 24 h, 1 μL to 1 mL of CellTracker green dye (final concentration 3 μM) was added to allow incubation in the incubator for 30 min to 40 min, and the cells were collected and counted for subsequent use. Labeling of macrophages: LPS (final concentration 100 ng/ml) and IFN-γ (final concentration 20 ng/ml) were added to RAW264.7 cells in 24-well plate and incubated for 24 h, the old medium was removed and the cells were washed with PBS 3 times, and FA-Lipo@Til NCs in Example 3 were added and incubated for 12 h; the cells were washed with serum-free culture medium, 200 μL of fresh complete medium was added, and 2 μL to 1 mL of CellTracker deep red dye was added to mark the cells, the cells could be gently blown to a suspended state during this period, and the macrophages were gently collected with a pipette after 30 min to 40 min and counted for later use. Macrophages and apoptotic cells were mixed and cultured: apoptotic cells were added to macrophages at a ratio of 1:3, cultured in a 12-well plate in serum-free medium for 4 h to 6 h, the cells were collected into a flow tube and centrifuged at 800 rpm for 3 min. The supernatant was discarded, the cells were washed with 2 mL PBS and centrifuged under the same conditions, and this process was repeated twice. Finally, the cells were resuspended in 200 μL PBS for testing; fluorescence detection was conducted using FCM. CellTracker green dye (Ex/Em=492/517, could be detected by flow cytometry FITC channel) was used to mark and detect apoptotic cells; CellTracker deep red dye (Ex/Em=630/660, could be detected by flow cytometry APC channel) was used to mark and detect macrophages. FIG. 22 shows that compared with Model (13.75±0.21) %, FA-Lipo@Til NCs had an approximately 1.95-fold enhancement in the ability to phagocytize apoptotic cells, proving that FA-Lipo@Til NCs could improve the uptake in macrophages, thereby more effectively playing a role in enhancing macrophage efferocytosis.

### (16) In Vivo Pharmacodynamic Evaluation

AS model mice and initial treatment plan: ApoE^{-/-} mice were fed a high-fat diet for 12 weeks and then randomly divided into 5 groups: Model group (normal saline group); Crude Til group (p.o., 4 mg/kg); Til NCs group (p.o., 4 mg/kg); Lipo@Til NCs group (p.o., 4 mg/kg); FA-Lipo@Til NCs group in Example 3 (p.o., 4 mg/kg). The Control group was replaced by C57BL/6J mice fed with ordinary diet in the same cycle. The drug was administered orally once a day for 12 consecutive weeks, ApoE--mice continued to eat a high-fat diet throughout the treatment, and body weight changes were recorded 2 times a week. After 12 weeks of continuous administration, the mice were fasted but allowed to drink water 12 h before the experiment. Their eyeballs were removed to collect whole blood serum, and then the mice were dissected, their heart was exposed, and a small incision was made in the right atrium, and the whole body was perfused with pre-cooled saline until the outflowing fluid became clear. The liver, spleen, lungs, and kidneys were collected, and the tissues around the heart and aorta were cleaned under a microscope to reveal the aorta and related three major branches (from left to right: brachiocephalic artery, left common carotid artery, and left clavicle). The heart and other parts were stored at −80° C., and the rest of the tissues were fixated with paraformaldehyde.

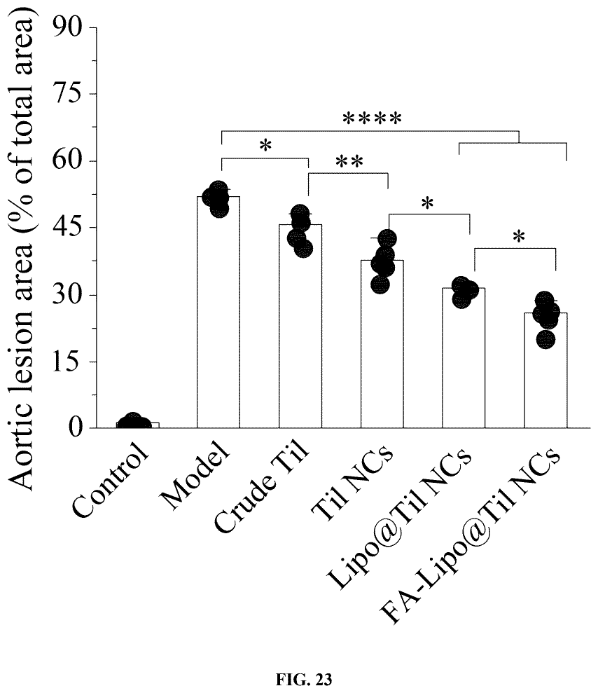

ORO staining of the entire aorta: fat tissue stained bright red by ORO could interfere with the observation of plaques, such that the fat tissue around the blood vessels needed to be removed with fine forceps under a stereomicroscope. The treated blood vessels were fixated with paraformaldehyde for 24 h and then removed. The aorta and its branches were longitudinally dissected by inserting spring scissors from the incision of the ascending aorta. The intima surface was unfolded and its shape was fixed with acupuncture needles. Preparation of ORO dye solution: 0.5 g powder was dissolved in 100 mL isopropanol, heated and ultrasonicated to obtain a saturated solution, which was mixed with double distilled water in a ratio of 3:2 to obtain a working solution, and both were stored in the dark. During staining, blood vessels were treated in working solution at 37° C. in the dark for 60 min, then differentiated in 60% isopropanol until plaques developed a color, and then terminated with distilled water. After photographing, the images were measured using ImageJ, and the percentage of plaques in the intima area was calculated to correct for differences in body size. FIG. 23 shows that compared with the Control group, the average plaque area of the aorta in the Model group (normal saline group) was 51.53%, indicating that the AS model was successfully constructed. However, after intragastric administration of Crude Til, the plaque area was reduced. In contrast, the Til NCs group (37.19%) reduced the average aortic plaque area by about 1 times compared with the Model group. More notably, the nanoparticles that were lipid-encapsulated and further FA-modified (Lipo@Til NCs and FA-Lipo@Til NCs) showed more significant effects, with the plaque areas reduced to 30.90% and 25.46%, respectively. In particular, the FA-Lipo@Til NCs group was about 2 times lower than that of the Model group, and its effect was significantly better than that of the Til NCs group.

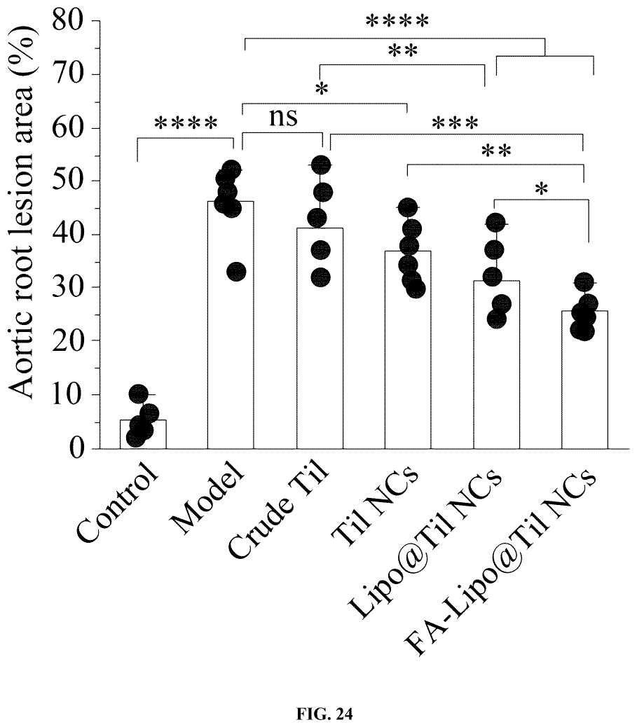

ORO staining of frozen sections of aortic root: after the heart was fixated for 24 h, it was processed and most of the apex cordis was cut off, and placed in an embedding box and immersed in October 4 μm thin sections were made with a freezing microtome at-20° C.; treatment of fresh frozen sections: the sections were rewarmed and dried, fixated with ice acetone, washed with water, filtered and then stained with ORO stain. The sections were differentiated with isopropanol and washed with pure water, then stained with hematoxylin and washed again. The sections were differentiated with alcohol, blued with distilled water, and then washed with tap water. The results were examined under a microscope and the sections were mounted. After sections were stained, they were photographed and the lipid content ratio of plaques was calculated using ImageJ. FIG. 24 shows that the average plaque area of the aortic root in the Model group was approximately 45.83%. This result confirmed the successful construction of the atherosclerosis model, and FA-Lipo@Til NCs (26.83%) had the most significant effect in reducing the average plaque area.

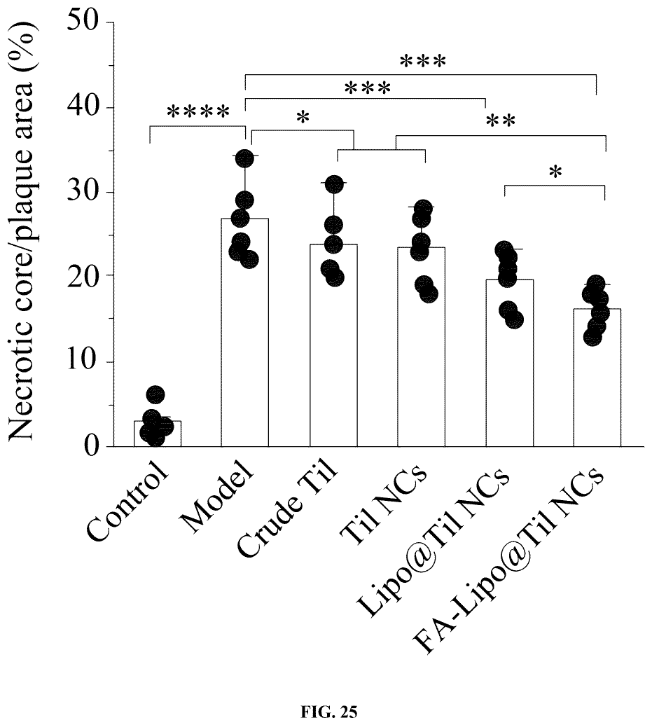

Hematoxylin-eosin (H&E) staining of frozen sections of aortic root: after 2-3 min of hematoxylin staining, the sections were differentiated with aqueous hydrochloric acid solution, blued with ammonia water, and washed with clean water. H&E staining made the nucleus appear purple-blue and the cytoplasm and matrix appear red. The sections were soaked in ethanol and n-butanol, transparentized with xylene, and then sealed with neutral gum, and images were scanned and analyzed with 3D HISTECH. FIG. 25 shows that in the Model group, the area of the necrotic region was approximately 26.50%, whereas the area of the necrotic core within the aortic root plaque in the atherosclerosis model mice treated with FA-Lipo@Til NCs decreased to 16.33%. This further confirmed the potential therapeutic effect of FA-Lipo@Til NCs in enhancing the stability of atherosclerotic plaques.

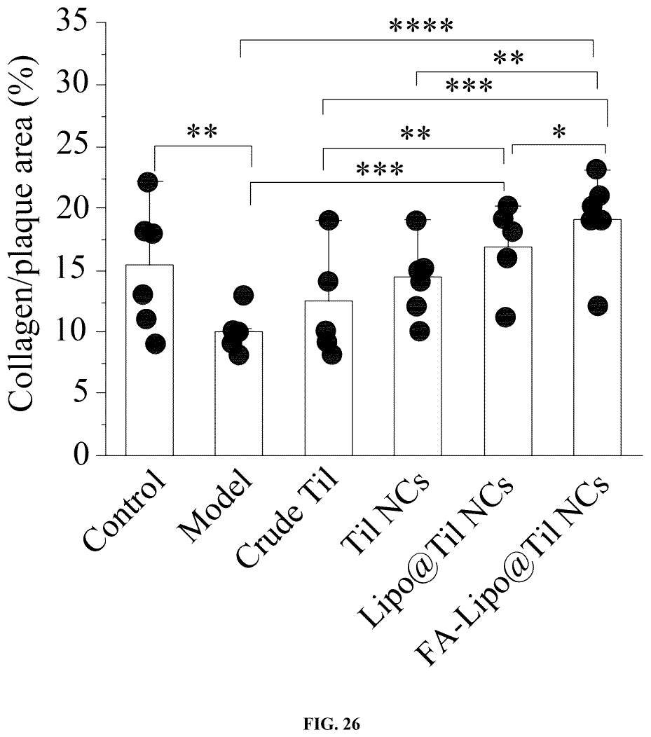

Masson staining of frozen sections of aortic root: the sections were treated with potassium dichromate, washed with water, immersed in freshly prepared iron hematoxylin stain for 3 min, rinsed with tap water, and briefly differentiated with hydrochloric acid alcohol. The sections were then stained with Ponceau acid fuchsin, treated with phosphomolybdic acid, and stained with aniline blue. The sections were differentiated with 1% glacial acetic acid, dehydrated with ethanol and n-butanol, and cleared with xylene. After being wiped clean with cotton swabs, the sections were mounted with neutral gum and dried in a fume hood. Scanning was conducted 1-2 days later to allow analysis. FIG. 26 shows that compared with the Control group (collagen content of 15.83%), the collagen content around the plaque in the Model group was significantly reduced to about 12.00%. However, compared with the Til NCs group, the collagen content around the plaques in the FA-Lipo@Til NCs group was significantly increased, indicating that FA-Lipo@Til NCs administered by oral gavage could stabilize the plaques and effectively inhibit the further development of atherosclerosis.

### (17) Evaluation of Inflammatory Factors in Serum

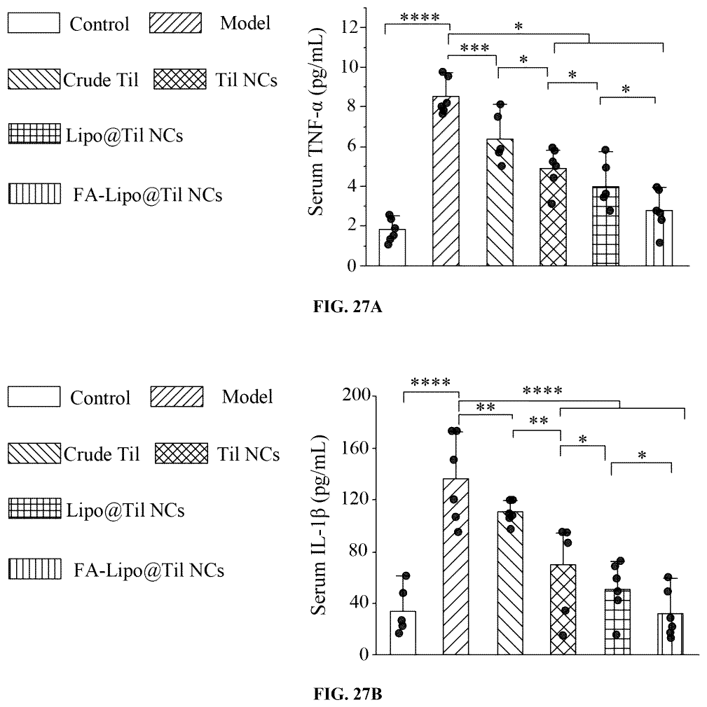

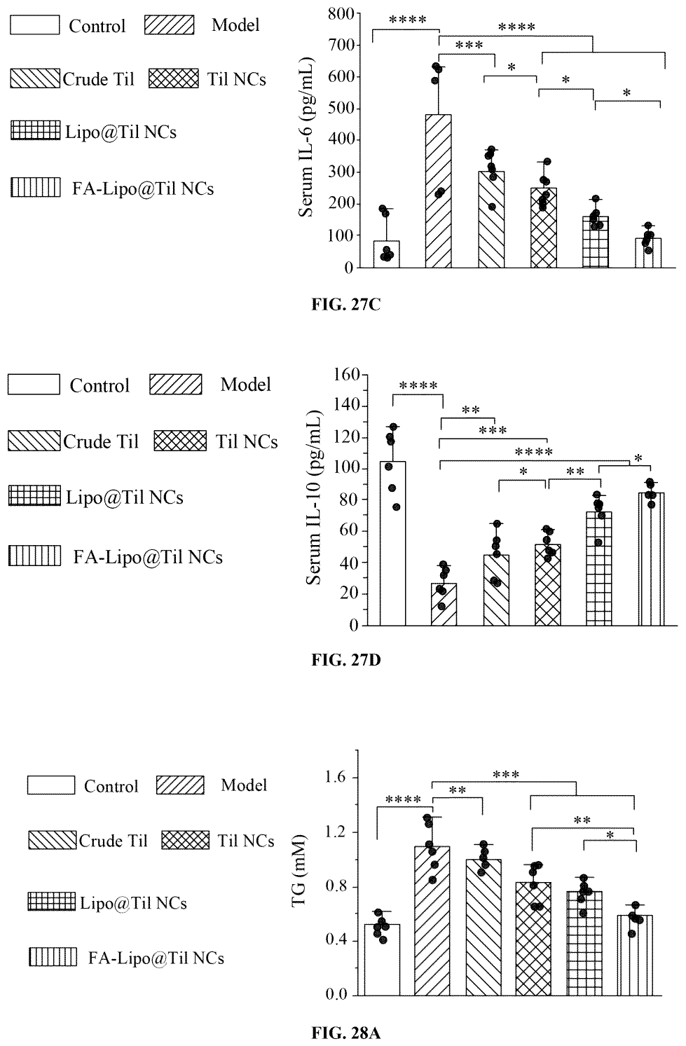

Blood samples from different treatment groups were collected, allowed to stand for coagulation, and then centrifuged to obtain the supernatant. The concentrations of TNF-α, IL-1β, IL-6, and IL-10 in serum were detected using an ELISA kit, and instructions for use of the kits were: TNF-α (cat: E-EL-M3063), IL-6 (cat: E-EL-M0044c), IL-1β (cat: E-EL-M0037c), and IL-10 (cat: E-MSEL-M0031). FIG. 27A-FIG. 27D show that compared with the Control group, the Model group significantly increased the levels of IL-6 (585.89 pg/mL), TNF-α (8.17 pg/mL), and IL-1β (150.48 pg/mL), and significantly decreased the level of IL-10 (34.12 pg/mL). This proved that the atherosclerosis model was successfully established. Compared with the other treatment groups (Crude Til, Til NCs, and Lipo@Til NCs), the expression of typical pro-inflammatory cytokines (IL-6, TNF-α and IL-1β) in the serum of FA-Lipo@Til NCs in Example 3 reached the lowest after treatment, while the expression of typical anti-inflammatory cytokine (IL-10) reached the highest. These results indicated that FA-Lipo@Til NCs treatment could significantly reduce the secretion of multiple inflammatory factors throughout the body.

### (18) Evaluation of Lipid Levels in Serum

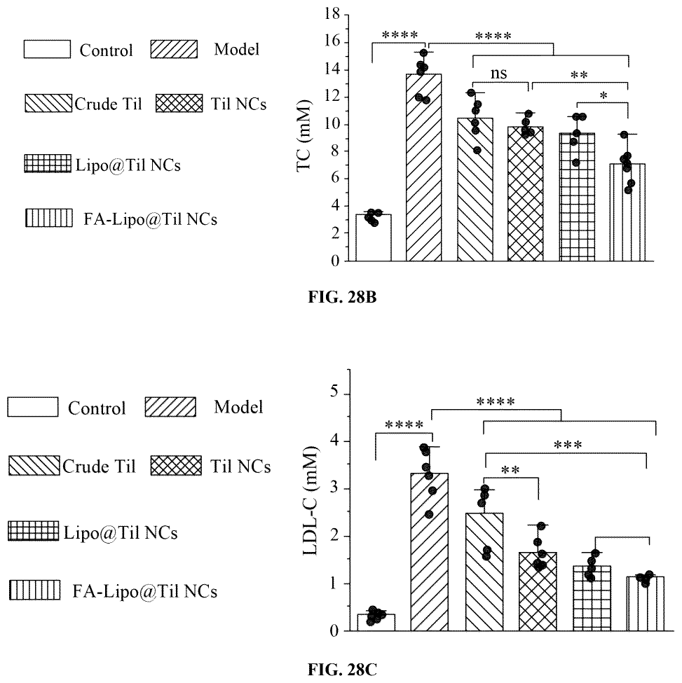

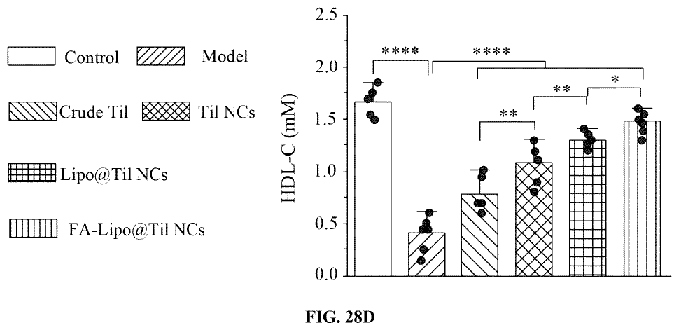

Blood samples from different treatment groups were collected, allowed to stand for coagulation, and then centrifuged to obtain the supernatant. Four blood lipid items were measured on an automatic biochemical analyzer: high-density lipoprotein cholesterol (HDL-C), low-density lipoprotein cholesterol (LDL-C), triglyceride (TG), and total cholesterol (TC). FIG. 28A-FIG. 28D show that compared with the Control group, the TG, CHOL and LDL levels in the serum of the Model group rats increased significantly, and the HDL level decreased significantly; compared with other treatment groups (Crude Til, Til NCs, and Lipo@Til NCs), the FA-Lipo@Til NCs in Example 3 had the lowest levels of TG, CHOL and LDL in serum after treatment, while its HDL content reached the highest. These results indicated that FA-Lipo@Til NCs treatment could significantly reduce the blood lipid levels of AS mice.

The above described are merely preferred implementations of the present disclosure rather than limitations to the present disclosure in any form. It should be noted that those of ordinary skill in the art may further make several improvements and modifications without departing from the principle of the present disclosure, but such improvements and modifications should be deemed as falling within the protection scope of the present disclosure.

What is claimed is:

1. A folic acid-modified liposome-encapsulated tilianin nanocrystal (FA-Lipo@Til NC), comprising a tilianin nanocrystal (Til NC) composition and a folic acid-modified phospholipid (FA-Lipo) bilayer encapsulated on a surface of the Til NC; wherein the Til NC composition comprises tilianin (Til) and a stabilizer; and raw materials of the FA-Lipo bilayer comprise phospholipid, cholesterol (Chol), a methoxy poly(ethylene glycol)-cholesterol conjugate (mPEG-Chol), and a folic acid (FA) compound, and the FA compound comprises FA and/or a FA derivative.

2. The FA-Lipo@Til NC according to claim 1, wherein the FA derivative is one or more selected from the group consisting of FA-polyethylene glycol (PEG)-Chol, 1,2-distearoyl-sn-glycero-3-phosphoethanolamine (DSPE)-PEG-FA, FA-PEG, FA-PEG-amine (NH2), FA-PEG-carboxylic acid (COOH), and FA-PEG-thiol (SH).

3. The FA-Lipo@Til NC according to claim 1, wherein the stabilizer comprises a suspending agent and/or a surfactant; the suspending agent is one or more selected from the group consisting of methylcellulose (MC), hydroxypropylcellulose (HPC), hydroxypropyl methylcellulose (HPMC), polyvinyl pyrrolidone (PVP), polyvinyl alcohol (PVA), PEG, sodium carboxymethylcellulose (CMC-Na), carbomer, dextran, and sodium alginate; and the surfactant is one or more selected from the group consisting of polysorbate 20, polysorbate 80, oleic acid, lauric acid, sodium deoxycholate, sodium taurocholate, sodium glycocholate, D-α-tocopherol polyethylene glycol succinate (TPGS), sodium lauryl sulfate (SLS), sodium hexadecyl sulfate (SHS), sodium octadecyl sulfate (SOS), sodium dodecyl sulfonate (SDS), polyoxyethylene castor oil, polyoxyethylene 40 hydrogenated castor oil, poloxamer 188, poloxamer 407, cetyl trimethylammonium bromide (CTAB), PEG (15)-hydroxystearate, and glyceryl monocaprylocaprate type I.

4. The FA-Lipo@Til NC according to claim 1, wherein the FA-Lipo@Til NC has a needle shape; and the FA-Lipo@Til NC has a particle size of 10 nm to 10 μm and a drug loading capacity of 5% to 99.9%.

5. A method for preparing the FA-Lipo@Til NC according to claim 1, comprising the following steps:
mixing the Til with a first organic solvent to obtain an organic phase;
mixing a stabilizer with water to obtain an aqueous phase;
mixing the organic phase with the aqueous phase, and then subjecting an obtained mixed solution to crushing and removal of the first organic solvent in sequence to obtain a suspension of the Til NC composition;
mixing the phospholipid, the Chol, the mPEG-Chol, and the FA compound with a second organic solvent, and then subjecting an obtained encapsulation layer solution to removal of the second organic solvent to obtain a milky white film; and
adding the suspension of the Til NC composition into the milky white film, and then subjecting an obtained mixture to hydration and an ultrasonic treatment in sequence to obtain the FA-Lipo@Til NC.

6. The method according to claim 5, wherein the organic phase and the aqueous phase are at a volume ratio of 1:5 to 1:50; the Til in the suspension of the Til NC composition has a mass concentration of 0.01 weight/volume percent (w/v %) to 80 w/v %; when the stabilizer is a suspending agent, the suspending agent in the suspension of the Til NC composition has a mass concentration of 0.01 w/v % to 20 w/v %; when the stabilizer is a surfactant, the surfactant in the suspension of the Til NC composition has a mass concentration of 0.01 w/v % to 10 w/v %; and
when the stabilizer comprises a suspending agent and a surfactant, the suspending agent has a mass concentration of 0.01 w/v % to 20 w/v %, and the surfactant has a mass concentration of 0.01 w/v % to 10 w/v % in the suspension of the Til NC composition.

7. The method according to claim 5, wherein the phospholipid has a mass concentration of 0.05 w/v % to 20 w/v %, the Chol has a mass concentration of 0.01 w/v % to 5 w/v %, the mPEG-Chol has a mass concentration of 0.002 w/v % to 1 w/v %, and the FA compound has a mass concentration of 0.0001 w/v % to 1 w/v % in the encapsulation layer solution.

8. The method according to claim 5, wherein the Til in the suspension of the Til NC composition and the phospholipid in the milky white film are at a mass ratio of 1:1 to 5:1; and the hydration is conducted for 10 min to 120 min, and the ultrasonic treatment is conducted at an ultrasonic power of 50 W to 900 W for 3 min to 120 min.

9. A method for treating a disease by promoting generation of an anti-inflammatory macrophage and repairing efferocytosis, comprising administering a drug comprising the FA-Lipo@Til NC according to claim 1 to subject in need thereof.

10. The method according to claim 9, wherein the disease is selected from the group consisting of myocardial ischemia-reperfusion injury (MIRI), acute lung injury (ALI), acute kidney injury (AKI), hypertension, myocardial infarction (MI), atherosclerosis, diabetes, cancer, allergic asthma, Parkinson's disease, a non-alcoholic liver disease, vascular dementia, or an inflammatory disease; and a dosage form of the drug is selected from the group consisting of an injection, an oral solution, an external ointment, a transdermal patch, a gel, a capsule, a drop pill, a drop, and a spray.
